(19) United States
(12) Patent Application Publication
(10) Pub. No.: US 2017/0159130 A1
(43) Pub. Date: Jun. 8, 2017

(54) TRANSCRIPTIONAL CLASSIFICATION AND PREDICTION OF DRUG RESPONSE (T-CAP DR)

(71) Applicants: Amit Kumar Mitra, Minneapolis, MN (US); Brian Van Ness, Minneapolis, MN (US)

(72) Inventors: Amit Kumar Mitra, Minneapolis, MN (US); Brian Van Ness, Minneapolis, MN (US)

(21) Appl. No.: 15/366,912

(22) Filed: Dec. 1, 2016

Related U.S. Application Data
(60) Provisional application No. 62/262,709, filed on Dec. 3, 2015.

(51) Int. Cl. C12Q 1/68; A61K 38/07; A61K 38/05

(52) U.S. Cl. CPC C12Q 1/6886; A61K 38/05; A61K 38/07; C12Q 2600/106; C12Q 2600/158

(57) ABSTRACT

A method to predict the response. e.g., a strong positive response or a strong lack of responsive, of myeloma cells of a multiple myeloma patient to a proteasome inhibitor is provided.

### CROSS-REFERENCE TO RELATED APPLICATIONS

This application claims the benefit of the filing date of U.S. application Ser. No. 62/262,709, filed on Dec. 3, 2015, the disclosure of which is incorporated by reference herein.

### BACKGROUND

Wide inter-individual variation in response to chemotherapy is a major limitation in achieving consistent therapeutic effect in many cancers, including multiple myeloma (MM), the second-most common hematologic malignancy with an estimated 30.330 new cases (about 2% of all new cancer cases) and 12,650 estimated deaths in 2016 (NCI-SEER Cancer statistics) (Marin et al., 2012; Richardson et al., 2007; Fonseca et al., 2009; Vangsted et al., 2011). Such heterogeneity in response to treatment is governed in large part by the underlying molecular characteristics of the tumor, including differences in the expression of genes involved in mechanisms of chemo-resistance (Kumar et al., 2008; Rajkumar et al., 2014; Rajkumar, 2016). Deciphering key changes in genes expression levels underlying personalized sensitivity to chemotherapy is therefore essential to predict the efficacy of anti-cancer drugs and to prevent delay in the selection of more effective alternative strategies.

Proteasome inhibitors/PIs are effective chemotherapeutic agents in the treatment of MM, used alone or in combination with other anti-cancer agents like alkylating agents, corticosteroids, immunomodulatory agents and histone deacetylase inhibitors (HDACis) Rajkumar, 2014; Mitsiades et al., 2011; Richardson et al., 2011). Bortezomib (Bz/Btz/Velcade) was the first proteasome inhibitor to be approved by US-FDA for clinical application in 2003 for the treatment of relapsed and refractory MM Moreau et al., 2012; Lightcap et al., 2000; Adams et al., 1999). Other examples include second generation PIs Carfilzomib (Cz/Cfz/Kyprolis). Oprozomib (Opz) and Ixazomib (Ix/MLN9708/Ninlaro) (Rajkumar, 2011; Moreau et al., 2012; Kortuem et al., 2013). However, MM still remains mostly an incurable disease with 5-year survival rate of 48.5% (NCI-SEER Cancer statistics). Further, most MM patients ultimately undergo relapse, including patients with good response to initial treatment who eventually develop resistance to the therapy (Rajkumar, 2016). Moreover, there are reports that patients who fail to respond to Bz may still respond to other PIs (Mitsiades et al., 2011; Rajkumar et al., 2013).

Most patients receive PIs in combination with other therapeutic agents: thus, the variability in PI response is difficult to assess. Additionally, survival endpoints in clinical applications are measured in months to years, so that developing prediction algorithms of response can be a long process.

### SUMMARY

No study thus far has attempted to combine multiple computer driven machine learning approaches on a substantially large training dataset to generate a gene expression profile (GEP) signature-based algorithm that can predict drug response in tumor cells. As described herein, human MM cell lines (HMCLs) were used as a model system and the proteasome inhibitor (PI) Bortezomib (Bz) as a model drug, in a predictive algorithm using machine learning approaches to forecast probability of PI response of individual tumors based on a targeted GEP signature. This approach was applied to create a drug response GEP signature from drug sensitivity profiles of cell lines from the Cancer Genome Project (about 600 cell lines). An automated computational process of prediction of drug response to a wide array of drugs based on the drug targeted classification transcriptome program (Drug Target CTP) can thus be developed.

In particular, a collection of more than 50 human mycloma cell lines/HMCLs generated through the immortalization of primary MM cells (PMCs), representing a broad spectrum of the biological and genetic heterogeneity of MM (Moreau et al., 2011), was used to create an in vitro chemosensitivity profile in response to treatment with four PIs: Bz. Cz. Ix and Opz as single agents. Then machine learning-based computational approaches were used to identify gene signatures that could predict PI-response in cell lines. When applied to gene expression data of MM patients undergoing PI-based clinical trials, the GEP model of response/resistance to PIs successfully predicted differences in disease progression and distinguished between extraordinary (good and poor) outcomes. Thus, these results provide a PI treatment-specific prediction. e.g., of clinically relevant outcomes that could impact therapeutic choices. Accordingly, use of the expression data (panel), e.g., to direct therapy, is provided. For example, a specific proteasome inhibitor may be indicated and another proteasome inhibitor may be counterindicated for a particular patient, or a specific proteasome inhibitor may be counterindicated when used alone but may be indicated if used in a combination therapy for a specific patient.

In one embodiment, a method is provided to predict strong response or strong resistance of myeloma cells to proteasome inhibitor therapies using gene expression analysis of a targeted panel of gene.

In one embodiment, use of a gene expression panel that provides a profile for a patient with multiple myeloma to predict proteasome inhibitor resistance or sensitivity, probability of progression, complete response (CR), good response, poor response, or no response, is disclosed. In one embodiment, a method to predict proteasome inhibitor response or resistance in a multiple myeloma patient is provided. For example, a profile from a gene expression panel from myeloma cells of the patient is provided. The profile includes gene expression from a plurality of genes that are upregulated or downregulated in a plurality of proteasome inhibitor sensitive multiple myeloma cell lines relative to corresponding expression in proteasome inhibitor resistant multiple myeloma cell lines. In embodiment, the profile assists in therapy selection. Foe example, one or more selected proteasome inhibitors are administered to the patient if the profile of the patient has a plurality of genes upregulated or downregulated that are upregulated or downregulated in the plurality of proteasome inhibitor sensitive multiple myeloma cell lines or a non-proteasome inhibitor anti-cancer drug or a combination of anti-cancer drugs is administered to the patient if the profile of the patient has a plurality of the genes that are upregulated or downregulated in the proteasome inhibitor resistant multiple myeloma cell lines relative to the plurality of in the proteasome inhibitor sensitive multiple myeloma cell lines. In one embodiment, the panel includes two or more genes that are upregulated or downregulated in one or more of ANBL6, ARP1, Arp1-1c, FLAM76, H1112, H929, JIM3, JJN3, JK67, Karpas620, Kas61, KHMB1, KMM1, KMS11, KMS12BM, KMS12PE, KMS18, KMS20, KMS26, KMS34, KP6, L363, LP1, MM1-144, MM1S, MMM1, MOLP8, OCIMY1, OCIMY5, OCIMY7, PE2, RPMI8226, Sachi, SKMM1, SKMM2, U266, UTMC2, XG1, XG2, XG6, or XG7. In one embodiment, the panel includes two or more genes that are upregulated or downregulated in one or more of AMO-1, ANBL6, ARD, ARP1, ARP1-1c, ARP-An1, DELTA47, FLAM76, FR-4, H1112, H929, JIM3, JJN3, JK6L, Karpas620, Kas6/1, KHM18, KMM1, KMS11, KMS128M, KMS12PE, KMS-20, KMS26, KMS28PE, KMS34, KP6, L363, LP1, MM1-144, MM1S.P, MMM1, MOLP2, MOLP8, OCI-MY1, OCI-MYS, OCI-MY7, OPM1, OPM2, PE2, RMPI8226, Sachi, SKMM1, SKMM2, U266.P, UTMC2, XG-1, XG-2, XG-6, or XG-7. In one embodiment, the panel detects RNA transcripts using two or more primers or probes to detect upregulation or downregulation of one or more of genes including but not limited to HIST1H2BD, PAQR6, NEK3, ZNFX1-AS1, C6orf48, LYSMD1, RNF170, HSPA18, TFEB, SOX12, POLR3GL, MNAT1, TARS2, UBE2K, PLK1S1, DLST, MYH9, C2orf69, GLA, RAB8A, SFMBT2, NCAPH2, SVIP, TUBA4A, MSL3, WFS1, MRI1, ABHD2, CERK, KHK, LMF2, PTPN18, SLC1A4, CYTIP, or AR. In one embodiment, the panel detects RNA transcripts using two or more primers or probes specific for expression of one or more of NEK3, DLST, PLK1S1, TARS2, C6orf48, HSPAIB, TFEB. RNF170, SOX12, ZNFX1-AS1, PAQR6, HIST1H2BD, UBE2K, LYSMD1, MNAT1, or POLR3G, wherein expression thereof is downregulated in proteasome inhibitor sensitive multiple myeloma cells. In one embodiment, the panel detects RNA transcripts using two or more primers or probes specific for expression of one or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2, the expression thereof is upregulated in proteasome inhibitor sensitive multiple myeloma cells. In one embodiment, the panel includes any combination of primers or probes for NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3G, SLCIA4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2, e.g., the panel includes probes for three, four, five, eight, ten, fourteen, fifteen, twenty, twenty-five, thirty, or thirty-five or more of those genes. In one embodiment, the panel does not include probes for CCND1 (cyclin-D1). HSPA1B, Gla, Wolfram syndrome 1/WFS1, X-box binding protein/XBP1, EB/TFEB, or RNF170, or any combination thereof.

In one embodiment, a method to predict drug response, e.g., to one or more proteasome inhibitors, in malignant plasma cells in a multiple myeloma patient is provided. The method includes providing an expression profile from multiple myeloma cells of the patient. When certain genes are upregulated or downregulated, relative to genes in proteasome inhibitor resistant myeloma cells, it is determined that the patient is sensitive to one or more proteasome inhibitors. In one embodiment, the patient is administered one or more of those proteasome inhibitors, e.g., based on their profile. In one embodiment, the proteasome inhibitor comprises bortezamib, ixazanib, carfilzomib or oprozomib. In one embodiment, the patient has relapsed myeloma. In one embodiment, the patient is newly diagnosed with myeloma. In one embodiment, the patient is relapsing. In one embodiment, the patient's disease is progressing. In one embodiment, the patient is non-responsive to an anti-myeloma treatment. In one embodiment, downregulation of one or more of NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3GL, or upregulation of one or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2, or any combination thereof, is indicative that the patient is sensitive to one or more proteasome inhibitors, or will have a good response to proteasome inhibitor treatment, e.g., >250 days until progression. In one embodiment, upregulation of one or more of NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3GL, or downregulation of one or more of SLC1A4, GLA, AKNA, ARHGAP27, LY96, MSL3, SQRDL, NCAPH2, MRI1, OBFC2A, RAB8A, ABHD2, LMF2. TUBA4A, C2orf69, PTPN18, PRKD2, KHK, CERK, GPSM3, WFS1, MYH9, CYTIP, SVIP. ARSA, or SFMBT2, or any combination thereof, is indicative that the patient is resistant to one or more proteasome inhibitors. In one embodiment, the panel lacks primers or probes, e.g., does not detect expression, for one, two three, four, five, six, seven, eight, nine, ten fifteen or twenty of genes including NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, POLR3GL, SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2. In one embodiment, the panel lacks probes or panels for one or more of NEK3, DLST, PLK1S1. TARS2, C6orf48, HSPA1B. TFEB, RNF170, SOX12, ZNFX1-AS1, PAQR6. HIST1H2BD, UBE2K, LYSMD1, MNAT1, or POLR3GL. In one embodiment, the panel lacks primers or probes for one or more of SLCIA4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2.

Also provided is a panel of primers or probes for gene expression profiling. The panel may include, in one embodiment, primers or probes for one or more of ANBL6, ARP1, Arp1-1c, FLAM76, H1112, H929, JIM3, JJN3, JK67, Karpas620, Kas61, KHMB1, KMM1, KMS11, KMS128M, KMS12PE, KMS18, KMS20, KMS26, KMS34, KP6, L363, LP1, MM1-144, MM1S, MMM1, MOLP8, OCIMY1, OCIMY5, OCIMY7, PE2, RPMI8226, Sachi, SKMM1, SKMM2, U266, UTMC2, XG1, XG2, XG6, or XG7. In one embodiment, the panel includes probes for one or more of AMO-1, ANBL6, ARD, ARP1, ARP1-1c, ARP-An1, DELTA47, FLAM76, FR-4, H1112, H929, JIM3, JJN3, JK6L, Karpas620, Kas6/1, KHM18, KMM1, KMS11, KMS128M, KMS12PE, KMS-20, KMS26, KMS28PE, KMS34, KP6, L363, LP1, MM1-144, MM1S.P, MMM1, MOLP2, MOLP8, OCI-MY1, OCI-MYS, OCI-MY7, OPM1, OPM2, PE2, RMPI8226, Sachi, SKMM1, SKMM2, U266.P, UTMC2, XG-1, XG-2, XG-6, or XG-7. In one embodiment, the panel includes primers or probes for two or more of HIST1H2BD. PAQR6, NEK3, ZNFX1-AS1, C6orf48, LYSMD1, RNF170, HSPA18, TFEB, SOX12, POLR3GL, MNAT1, TARS2, UBE2K, PLK1S1, DLST, MYH9, C2orf69, GLA, RAB8A, SFMBT2, NCAPH2, SVIP, TUBA4A. MSL3, WFS1, MRI1, ABHD2, CERK. KHK, LMF2. PTPN18, SLCIA4, CYTIP, or AR. In one embodiment, the panel lacks primers or probes for one, two three, four, five, six, seven, eight, nine, ten fifteen or twenty of the following genes: NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, POLR3GL, SLCIA4, GLA, AKNA, ARHGAP27, LY96, MSL3, SQRDL, NCAPH2, MRI1, OBFC2A, RAB8A. ABHD2, LMF2, TUBA4A, C2orf69, PTPN18, PRKD2, KHK, CERK, GPSM3, WFS1, MYH9, CYTIP, SVIP, ARSA, or SFMBT2. In one embodiment, the panel lacks primers or probes for one or more of NEK3, DLST, PLK1S1, TARS2, C6orf48, HSPA1B. TFEB, RNF170. SOX12. ZNFX1-AS1, PAQR6, HIST1H2BD, UBE2K, LYSMD1, MNAT1, or POLR3GL. In one embodiment, the panel lacks probes or primers for one or more of SLC1A4, GLA, AKNA, ARHGAP27. LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2. In one embodiment, the probes are affixed, e.g., covalently attached, to a substrate in a microarray format. The primers or probes may be employed to predict time to progression of multiple myeloma patients, e.g., administered one or more proteasome inhibitors.

Further provided is a method to predict time to progression in a multiple myeloma patient administered a proteasome inhibitor. The method includes detecting gene expression for a panel of genes from myeloma cells of the patient, thereby providing a profile of gene expression in the multiple myeloma cells of the patient, wherein the panel includes a plurality of genes that are upregulated or downregulated in proteasome inhibitor sensitive multiple myeloma cell lines exposed to one or more proteasome inhibitors relative to corresponding expression in proteasome inhibitor resistant multiple myeloma cell lines; and determining if two or more of the following genes are upregulated or downregulated in the profile of the patient: NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, POLR3GL, SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2. If one or more of SLCIA4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2 is upregulated and one or more of NEK3, DLST, PLK1S1, TARS2, C6orf48, HSPA1B. TFEB, RNF170, SOX12, ZNFX1-AS1, PAQR6, HIST1H2BD, UBE2K, LYSMD1, MNAT1, or POLR3GL is down regulated, or two or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK. CERK, GPSM3, WFS1, MYH9, CYTIP, SVIP, ARSA, or SFMBT2 is upregulated, or one or more of NEK3, DLST, PLK1S, TARS2, C6orf48, HSPA1B, TFEB, RNF170, SOX12, ZNFX1-AS1, PAQR6, HIST1H2BD, UBE2K, LYSMD1, MNAT1, or POLR3GL is downregulated, in the profile of the patient, the patient has a longer time to progression relative to a patient with a profile where SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, and SFMBT2 are upregulated and NEK3, DLST, PLK1S1, TARS2, C6orf48, HSPA1B, TFEB, RNF170, SOX12, ZNFX1-AS1, PAQR6, HIST1H2BD, UBE2K. LYSMD1, MNAT1, and POLR3GL are downregulated, or where SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2 are upregulated, or NEK3, DLST, PLK1S1, TARS2, C6orf48, HSPAIB, TFEB, RNF170, SOX12, ZNFX1-AS1. PAQR6. HIST1H2BD, UBE2K, LYSMD1. MNAT1, and POLR3GL are downregulated. In one embodiment, a microarray is employed to detect gene expression. In one embodiment, RNA sequencing is employed to detect gene expression.

### BRIEF DESCRIPTION OF THE DRAWINGS

FIG. 1. A comparative analysis of in vitro chemosensitivity to 4 PIs among HMCLs.

FIG. 2. A heat map generated using unsupervised hierarchical clustering (HC) analysis for most differentially expressed genes (p<0.001) between HMCLs with Top-5 vs Bottom-5 Bz IC₅₀ values.

FIG. 3. Kaplan-Meier curves showing significant differences in OS in patients (Top vs Bottom 20% survivors) from the Bz of APEX trial clustered on the basis of the expression of the genes that most distinguished Bz-Sensitive and Bz-Resistant cells lines.

FIG. 4. A schematic representation of analysis workflow: R Package that automates classification, predication and quantitation of drug sensitivity of individual test sample for multiple drugs, using Cancer genome project drug-cell line data as training set.

FIGS. 5A-F. In vitro chemo-sensitivity profiles of human mycloma cell lines following proteasome inhibitor treatment. A-D) Plots show survival compared to untreated control versus increasing concentration of bortezomib, oprozomib, ixazomib, carfilzomib. In E) the Area Under the Survival Curve (AUSC) was normalized and expressed as percentage of the largest value for each drug, shown for all cell lines treated with the four proteasome inhibitors. In F) the Scatterplot matrix is shown as a pairwise correlation of the natural log (Ln) of IC₅₀ and AUSC values for the response to each PI drug. Scatterplot matrix was generated using the R graphing package ggplot2.

FIG. 6. Heat map representing differential gene expression between PI-sensitive vs PI-resistant myeloma cell lines. Gene expression was z-score normalized (standardized: shifted to mean of 0 and scaled to standard deviation of 1) and compared among the 5 most Ix-responsive and 5 least Ix-responsive cell lines. Heatmap was generated using the top 42 differentially expressed genes (|fold-difference|>2; p<0.01). Columns are ordered by Ix IC₅₀ of cell; genes are ordered by fold-difference. Color indicates fold-change between Ix-resistant vs Ix-sensitive cell lines.

FIGS. 7A-F. Plots showing stratification in progression-free survival (PFS) among MM patients on PI-based clinical trials in which the 42-gene model was used to assign extraordinary (good and poor) PI-response. Kaplan-Meier survival curves in A-B) APEX dataset: bortezomib arm shows significant separation of PFS between clusters representing good vs poor outcomes; whereas the dexamethasone arm shows no stratification; C-D) patients in the HOVON-GMMG-HD4 trial (Bz-treated/PAD and VAD arms) were assigned good versus poor PI-response based on the 42 gene model. PFS curves for the interim analysis of the E) CoMMpass trial (NCT0145429) patients administered Bz as first line of therapy, and F) the Mayo Clinic Ix-trial (NCT01415882). Patients were assigned good versus poor response based on the 42-gene model. Inset of 7F) shows survival of each patient considered. Dashed line represents end of year 1 (365.25 days from randomization).

FIGS. 8A-B. Ingenuity Pathway analysis showing the prediction of the A) top network and B) top upstream regulator based on the 42-gene signature of PI-response. Gene nodes are displayed using various shapes denoting the functional class of the gene product. Green symbolizes downregulation of gene expression, while red represents upregulation of gene expression. The significance of up/down-regulation is represented by color intensity. The IPA upstream regulator analysis identified TFEB and CCND1 as the top hits/factors that may control the genes and pathways highlighted by network analysis.

FIG. 9. Flow chart.

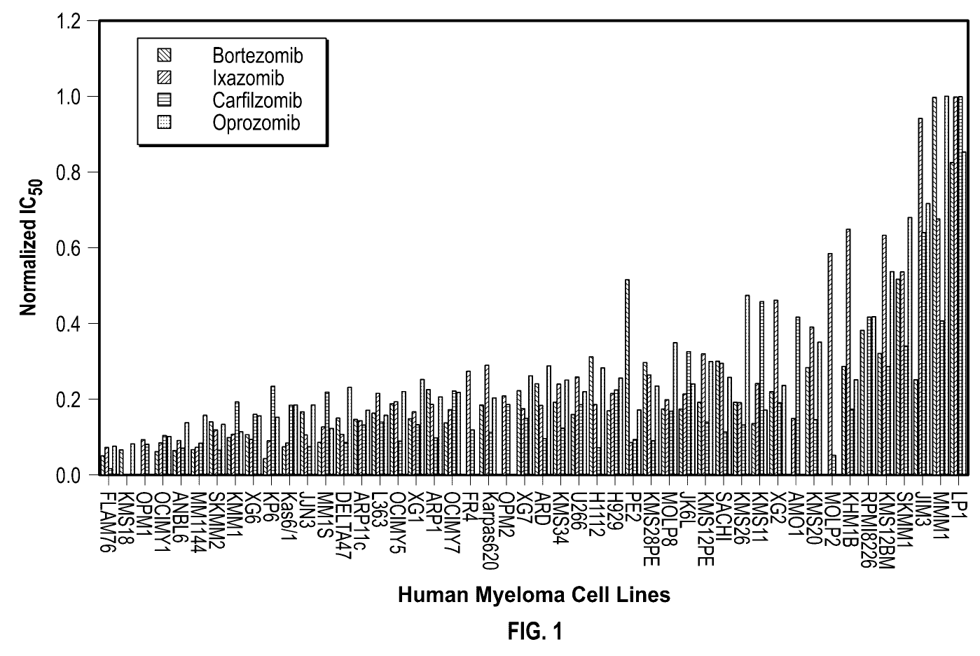

### DETAILED DESCRIPTION

The successful application of predictive algorithms for drug response in cancer can improve therapeutic outcomes by correlating each individual tumor expression profile to an effective cancer drug. Extensive inter-individual variation in response to chemotherapy is a major stumbling block in achieving desirable efficacy in the treatment of cancers, including multiple myeloma (MM). In this study, our goal was to develop a gene expression signature that predicts response specific to proteasome inhibitors (PI) treatment in MM. Using a well-characterized panel of human myeloma cell lines (HMCLs; n=50) representing the biological and genetic heterogeneity of MM, an in vitro chemosensitivity profile was identified in response to treatment with the four PIs Bortezomib (Bz), Carfilzomib (Cz), Ixazomib (Ix) and Oprozomib (Opz) as single-agents was identified. Gene expression profiling was performed using next-generation high-throughput RNA-sequencing. Applying machine learning-based computational approaches including the supervised ensemble learning methods Random forest and Random survival forest, a 42-gene expression signature was identified that could not only predict PI-response in the HMCL panel, but could also be successfully applied to four different clinical datasets on MM patients undergoing PI-based chemotherapy to predict PI-response as well as to distinguish between extraordinary (good and poor) outcomes. The results demonstrate the use of in vitro modeling and machine learning-based approaches to establish predictive biomarkers of response and resistance to drugs that may serve to better direct myeloma patient treatment options.

In a first embodiment, a signature subset of genes highly correlated with baseline Bz response or resistance using the machine learning approaches was generated (feature selection). Gene expression analysis and in vitro chemosensitivity data on Bz from Cancer Genome Project (n˜600 cell lines) will be obtained. From there, a classification model can be built, generate a GEP-based signature profile of baseline Bz response (Drug Target CTP) from drug sensitivity-gene expression analysis data on cell lines using a combination of Random Forest and Least Absolute Shrinkage and Selection Operator (LASSO) methods (feature selection).

A second embodiment predicts probability of PI response in a panel (n=50) of human myeloma cell lines (HMCLs) based on the feature selection generated/discussed above (first embodiment). Gene expression and in vitro chemosensitivity data on Bz for the HMCL panel (n=50) is obtained. One embodiment predicts the probability of PI response in the panel of human myeloma cell lines (HMCLs) based on the feature selection using a combination of Random Forest. SVM (radial) and SVM (sigmoidal) methods and using an Ensemble forecasting algorithm. The predictions of PI response are correlated with in vitro Bz chemosensitivity data.

A third embodiment predicts the probability of drug response of patient tumor samples the feature selection generated/discussed above (first embodiment). Gene expression analysis and response data of patient samples (treated with proteasome inhibitors) are obtained. One embodiment predicts the probability of PI response of individual patients using the Ensemble forecasting algorithm from the second embodiment. The predictions of PI response are correlated with patient outcomes (Response, OS, PFS).

A fourth embodiment will develop an R package based on the analysis pipeline that will automate the process of building binary classification models, selecting a set of genes (Drug Target CTP) for each of the drugs profiled in CGP followed by prediction of probability of drug response for each individual drug in CGP test samples (cell lines) based on the process of the first through third embodiments discussed above. This approach will enable the goal of improving therapeutic strategies by GEP based targeted response predictions to a wide variety of drugs.

Conventional unsupervised learning methods, like principal component analysis (PCA) and hierarchical clustering (HC) analysis, cannot make quantitative predictions for probability of drug sensitivity or resistance of individual tumors. In contrast, supervised machine learning approaches construct algorithms that learn from data, build a model from training inputs and make data-driven predictions/decisions on new, test samples (Hastie et al., 2009). As described below, algorithms have been demonstrated that used GEP signatures that successfully predict Bz response in myeloma cell lines and patients. The Cancer Genome Project provides a comprehensive database of drug-cell-line combinations that incorporates genomic and gene expression analysis data on 639 human tumor cell lines, representing a wide spectra of adult and childhood cancers of epithelial, mesenchymal and hematopoietic origin and drug-sensitivity data on 130 drugs covering a wide range of targets and processes involved in cancer biology (Garnett et al., 2012).

In 2014, National Cancer Institute (NCI) created the Dialogue on Reverse Engineering Assessment and Methods (DREAM) project and developed the NCI-DREAM drug sensitivity prediction challenge for the identification of top-performing methods for predicting therapeutic response based on genomic, proteomic, and epigenomic profiling data derived from a limited set of breast cancer cell lines (n=35 to build the model; n=18 to test the models). Among the 44 prediction algorithms that were developed, the top-performing approaches modeled nonlinear relationships and included machine learning approaches for drug sensitivity prediction. It was observed that in all the algorithms, drug response was modeled as a continuous outcome and prediction algorithms mostly focused on regression modeling of chemosensitivity data that attempted to rank order response of all the test lines. None achieved complete accuracy. With a large dataset of greater than 600 cell lines in the CGP, a classification model-based, machine learning approach can be undertaken where the focus will be on using the top-sensitive and top-resistant cell lines to estimate the probability of binary outcomes (sensitive vs resistant) in test samples. The outcome will provide a highly significant prediction of the most sensitive (25%, Q1) and most resistant (25%. Q4) tumor responses. This is an attainable and clinically significant outcome that can have a major impact on many patients being considered for specific drug therapies. This will provide a very useful tool/approach to those patients that will benefit most or least to a given treatment. Given the wide array of drug choices, alternative therapies, based on successful GEP predictions will improve outcomes. Herein is provided a model computational approach to improve diagnostic predictions, called the Drug Targeted Classification Transcriptome Program (Drug Target CTP).

As a first iteration, using a panel of 50 human myeloma cell lines, a computational approach combining multiple machine learning algorithms with the Ensemble forecasting approach was applied, and successfully developed a predictive GEP algorithm that was very effective in stratifying MM patient outcomes in clinical trials (Stressman et al., 2013: see below). The computational approaches can be applied to a wider array of cancer cell lines to develop highly predictive algorithms using targeted GEP signatures for a wider array of drugs. The modeling approaches described herein will have an impact in better identifying patient tumor responses, and improving outcomes based on the use of molecularly driven diagnostic selection of drugs. As demonstrated herein, such approaches can be very effective in selection of drugs in tumor samples that arise in emerging relapses (Stressman et al., 2013; Stressman et al., 2014). It has been further demonstrated that targeted GEP profiles can identify sensitive/resistant subpopulations by applying single cell targeted transcriptomics. Such an application, SCATTome (Single Cell Analysis of Targeted Transcriptome) has been developed (Mitra et al., 2016). Thus, the computational approaches can add significant value to more effective therapeutic designs that focus on individualized profiles.

The success in developing predictive GEP-driven algorithms on a collection of human myeloma cell lines was achieved by creating a pipeline to use the cytotoxic response to one drug, bortezomib (Bz), and correlated GEP, with the creation of a computational system for building binary classification model and feature selection, followed by prediction of probability of drug resistance using machine learning approaches that incorporated a combination of LASSO, Random Forest and Support Vector Machine (SVM) and Ensemble methods. The application of this approach can be used on a larger, more diverse cancer cell line collection, and a wide array of drugs. This innovative diagnostic approach will profile individual cancer samples using GEP, and make informed predictions of response to a wide array of drug choices. This will allow for computational approaches that ultimately meet the challenge of the Precision Medicine Initiative. The CGP cell lines will be used and the capabilities of predictive profiling against the myeloma-specific targets already established will be compared. Moreover, available gene expression analysis data and outcomes from MM patients as disclosed herein demonstrates the predictive accuracy in the clinical setting.

Molecular profiling, thus has the potential to significantly impact individualized therapeutic choices across oncology practice/wide range of cancers.

The following examples are intended to further illustrate certain particularly preferred embodiments of the invention and is not intended to limit the scope of the invention in any way.

### Example I

Plasma cell tumors in the mouse have been successfully modeled that show several features of human myelomas with similarities in genetic features that alter growth and response to drugs (Boylan et al., 2002). The laboratory has expertise in creating clonally-derived Bz-resistant cell lines from Bz-sensitive cell lines which has been documented in recently published literature (9). In recent years, in vitro modeling systems of MM have been developed for the identification of novel pharmacogenetic signatures predictive of baseline Bz response and kinetic Bz response in MM which was successful in stratifying good versus poor outcomes in human subjects, and could predict secondary therapies for proteasome inhibitor-based drug response (Stressman et al., 2013). The inventors also have a strong background in pharmacogenomic analysis in cancer treatments, and further expertise in informatics system development. Novel platforms to modeling human myeloma have been provided, that can be extended to other cancers.

Cytotoxic response of 50 human myeloma cell lines (HMCLs) to 4 different proteasome inhibitors (PIs) Bortezomib (Bz). Ixazomib (Ix) Carfilzomib (Cz) and Oprozomib (Opz) were profiled. PIs have become the backbone of common drug regimens in MM; however, extensive variability in patient responses is noted (non-responders as well as non-responding relapses) that were reflected in the variability of response seen in the HMCLs.

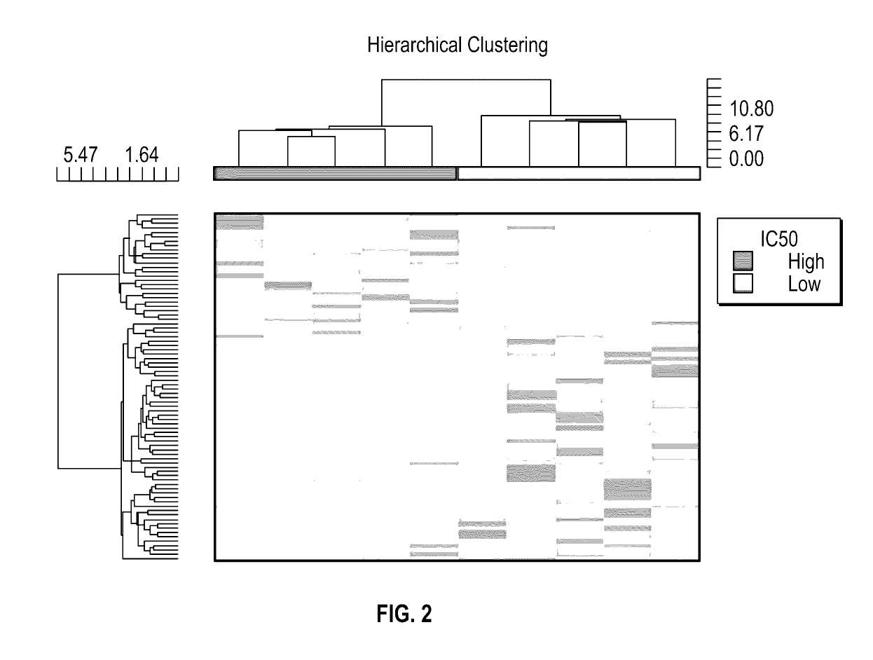

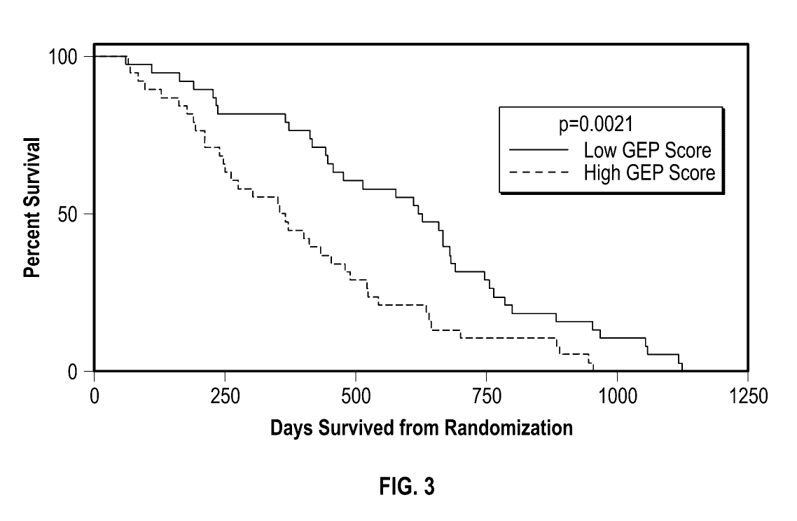

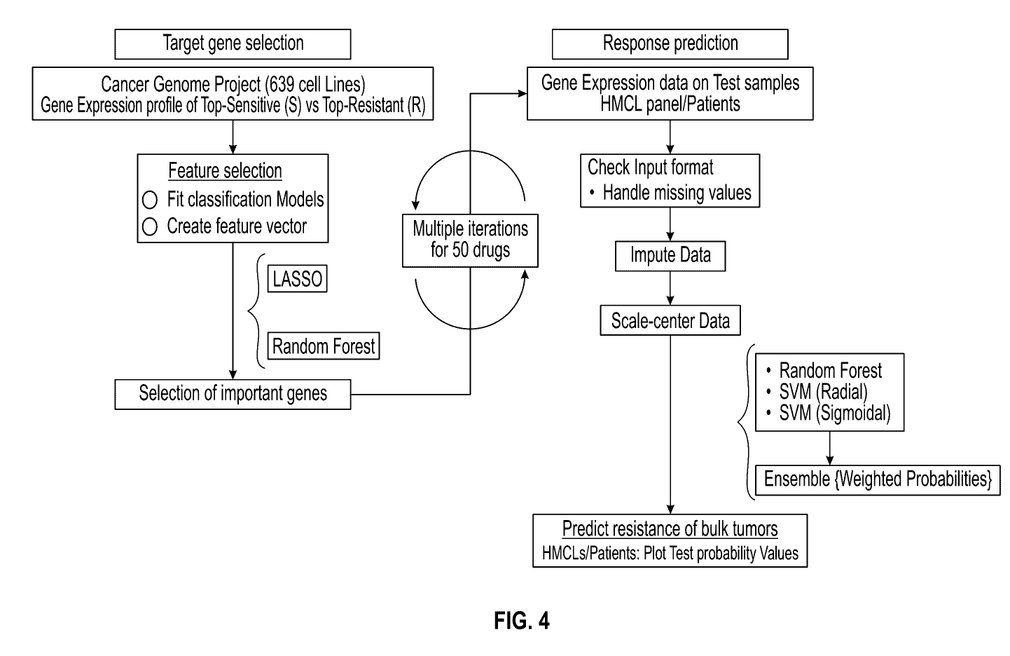

GEP profiles have been created for the HMCLs, and profiles of the most sensitive and most resistant cell lines were compared to bortezomib (Bz) and Ixazomib (Ix). More traditional hierarchical clustering identified a collection of differentially expressed profiles (FIG. 2).

Computational approaches were extended to use a combination of the machine learning methods LASSO and Random Forest and Support Vector Machine to identify a robust signature of PI response. Application of a response signature algorithm was very successful in stratifying good and poor response in patients on the APEX clinical trial (single agent Bz) (Stressman et al., 2013). It was further demonstrated that targeted transcriptomic profiling can be applied to single cells, and reveal subpopulation diversity of response that correlates with both residual populations that survive in vitro cytotoxic response, as well as patient depth of response. The application of computational approaches described herein can be applied to single cell analyses that would add further applications of developed algorithms of response.

The Cancer Genome Project contains a large collection of 639 human cancer cells of multiple types that integrates detailed genomic and gene expression analysis data with drug-sensitivity data (the half-maximal inhibitory concentration (IC₅₀)) on 130 drugs, including proteasome inhibitors Bortezomib and Carfilzomib (Garnett et al., 2012). The drugs screened include a wide spectrum of targets and processes involved in cancers, including targeted agents and cytotoxic chemotherapeutics that are already approved for clinics (n=31), drugs currently in various phases of clinical trials (n=47) and also experimental tool compounds (n=52). A total of 48,178 drug-cell-line combinations were screened with a range of 275-507 cell lines per drug. Herein below is a discussion to first develop predictive algorithms of Bz response in the CGP cell line panel as has been done with the myeloma cell line panel (HMCLs), build a classification model that is tested on the well characterized HMCLs and patient samples, then expand to an automated computational approach to create a GEP classification signature for 50 of the drugs profiled in CGP.

First, CGP data on gene expression profiles and in vitro cytotoxicity measures (IC₅₀) to Bz are downloaded. Next, an R statistical analysis package is develop as has been done for the myeloma cell lines. The pipeline will have functions to structure the data into a matrix, handle missing data with imputation, perform scale-centering, build classification models and select important genes (features) that strongly distinguish the top 60 responding cell lines from the bottom 60 (least responsive) cell lines. A subset of genes expressed (feature selection) that highly correlates with response with be created. Two statistical model-based approaches for feature selection. Least Absolute Shrinkage and Selection Operator (LASSO) and Random Forest, will be used. The LASSO operator selects genes while estimating a regression model simultaneously by minimizing a penalized likelihood of the data. The likelihood is penalized for the sum of the absolute values of the regression coefficients β estimates to force regression coefficients of the relatively non-significant genes to zero. The LASSO model minimizes the following penalized likelihood function (12):

$${min}_{β} {\sum }_{\{ i = 1 \}}^{N} {( {y}_{i} - {\sum }_{\{ j = 1 \}}^{p} {x}_{ij} {β}_{j} )}^{2} + λ {\sum }_{\{ j = 1 \}}^{p} \vert {β}_{j} \vert$$

Random forest is a decision tree based machine learning method that works by constructing a number of decision trees by random splitting of the data in its feature space and predicting the modal class in classification and the mean prediction in regression (Breiman et al., 2001). One of the advantages of the random forest method is that it provides a feature ranking based on importance of the genes in predicting the response (PI resistance of single cells in this case). While LASSO performs well as a feature selector, it may over-shrink the feature space thereby deselecting a few of the genes which may be biologically important in explaining drug resistance. Also, LASSO provides a subset without an importance ordering directly. Therefore, a triangulation of two models LASSO and Random Forest will be used for the final gene list selection. The intersection of the LASSO and the top genes from the importance ranked gene list provided by the Random Forest model will be used to create the final list for prediction.

Additionally, the gene expression profiles and in vitro cytotoxicity (IC₅₀) of the HMCLs (n=50) will be downloaded. The classification model will be tested based on the feature selection from above. For prediction of drug response, the set of important genes will be used as predictors to predict the probabilities of resistance for HMCLs. The prediction will be performed using an assortment of three different machine learning methods—Random Forest, Support Vector Machine (SVM) with Gaussian radial basis function (RBF) kernel and Support Vector Machine (SVM) with Hyperbolic Tangent (Sigmoid) Kernel, using the final selected genes (features) as predictors of the response variable. Support vector machines work by finding the best separator hyper-plane between two classes of objects (Steinwart et al., 2008). For all the prediction models, the average bootstrap prediction error will be generated using repeated bootstrapping of the train dataset and using a k-fold cross validation with k=100. The cross validation error rate will serve as a measure of the accuracy of the method. Two different kernels for SVM prediction will be used because SVM prediction is sensitive to the choice of kernels (Steinwart et al., 2008).

Error rates of each method will be used as weight factor for the predictions to generate a combined prediction from the three methods using Ensemble forecasting algorithm, as per the following equation:

$$\overline{p} \frac{{\sum }_{\{ i = 1 \}}^{3} \frac{{p}_{i}}{{e}_{i}^{2}}}{{\sum }_{\{ i = 1 \}}^{3} \frac{1}{{e}_{i}^{2}}}$$

where p is the prediction probability. The final fitted model will be used to generate predictions for the test set. Receiver operating characteristic (ROC) curves will be generated to visualize the performance of the binary classifier system as the discrimination threshold is varied over a range (Hanley et al., 1982). A classifier with a high area under the cytotoxic survival curve (AUC) is considered more preferred classifier (Brdley et al., 1997). The cytotoxic and GEP data has already been created for all the HMCLs. The drug responses obtained from prediction model estimation will be compared to experimental IC₅₀ data in the top 10 responding and bottom 10 nonresponding cell lines in order to determine the prediction accuracy of the gene expression signature associated with response/resistance.

The predictive GEP classifier on patient tumor cells will be further tested in a clinical trial using proteasome inhibitor therapy (Mayo Clinic trial MC1181) (n=58).

The gene expression profiles created by the Mayo Clinic on CD138+ sorted myeloma tumors from consented patients will be downloaded. Patient outcomes will also be provide as ECOG response criteria, overall survival (OS) and progression free survival (PFS). Gene expression analysis data will be analyzed using the predictors (features/gene signature) derived as discussed above, and the Ensemble approach will be used to predict the patient outcomes based on gene expression patterns. Based on previous modeling approach in the HMCLs, it is expected the classifier will provide significant predictive power in distinguishing the top 20% of responders from the bottom 20% non-responders.

The above applies the classification and feature selection on CGP cell lines to a specific test set in myeloma cell lines and patient samples.

Next the system will be automated in an R Package that will sort the cytotoxic response of the CGP cell lines to the array of drugs tested in the CGP Program and build classification models and perform feature selection for each drug from the CGP GEP database. The success in developing an R Package (Single Cell Analysis of Targeted Transcriptome/SCATTome) for single cell analysis in the myeloma studies allows for the automated drug response prediction for each cell. SCATTome scores, machine learning-based quantitative prediction scores of the probability of Bz resistance of single cells, using gene expression analysis data on 528 individual cells from 11 HMCLs and 418 individual cells from 8 drug naïve MM patient samples can be assembled. Similarly, automation of the classification using responder/non-responder GEP profiles for each selected drug in the CGP Program is performed.

The goal is to select 50 drug response profiles that most separate responder cell lines and non-responder cell lines—drugs that show the maximum inter-individual variation in response profiles, with priority to FDA approved or clinical trial drugs. For each drug, the top 10% responding CGP cell lines and bottom 10% non-responding lines (with 10 samples in each left out for test validation) will be identified. Classification models will be developed and GEP feature selection will be performed to predict drug response, as discussed above, and test the predictive power of our classifier on withheld samples. The resulting predictive algorithms for all the drugs will be developed into an R package of Transcriptional Classification of Drug Response (Transcript CDP), available on cran.r-project.org with GNU General Public License (GPL) version 3: a free software license, and a copyleft license.

Based on the successful predictive algorithms, a smaller cell line panel of human myeloma cell lines (n=50) was developed. A larger data set, e.g., of 600+ cell lines, provides a robust assessment of response variation to a wide array of drugs. The TCGA database (2008), and cooperative group clinical trial data (e.g., ECOG) may provide opportunities to explore outcome and genomic data bases. By providing the Transcript CDP as public access, it will provide investigators who have access to targeted cancer subtypes, drug responses, and expression profiles unique opportunities to explore predictive applications. Further, once established, the R package is easily applicable to new drug-cell line data sets that investigators (including Pharma) can apply.

As the initial focus herein has been in myeloma therapies, what we have established will be used to predict effective therapies using drugs that have defined predictive profiles that overlap with various subsets of myeloma tumors. This could be an effective strategy to 1) identify new, re-purposing of drugs used in other cancers, 2) identify drugs that may serve as secondary therapies after first-line relapses, 3) identify subpopulation sensitivities that may direct effective front-line drug combinations.

### Example II

### Materials and Methods

### Drugs

Bz (Takeda) was dissolved in serum-free RPMI-1640 (Lonza) and stored at −20° C. Ix (Takeda), Cz and Opz (Amgen) were dissolved in dimethyl sulfoxide/DMSO (Sigma) and stored at −20° C.

### Cell Lines

Fifty (50) HMCLs were procured from various institutions, established and characterized, and maintained in Human Myeloma Cell Line (HMCL) media with IL-6 (Mitra et al., 2016). Table 1A provides the cytogenetic characteristics of the HMCLs.

### In Vitro Chemosensitivity Assays

Cell cytotoxicity assays were performed on the HMCLs to create a drug sensitivity profile in response to treatment with increasing concentrations of Bz, Cz, Ix and Opz as single agents and half-maximal inhibitory concentration/IC₅₀ values and area under the survival curve/AUSC were calculated as described in Mitra et al. (2016). Briefly, cells were counted using Countess automated cell counter (Invitrogen) and seeded in 96-well plates at a concentration of 4×10⁵ cells per mL. After 24 hours, cells were treated with increasing concentrations of Bz, Cz, Ix and Opz as single agents. Cell viability assays were performed 48 hours post treatment using CellTiter-Glo luminescent cell viability assay (Promega) according to manufacturer's instructions using Synergy 2 Microplate Reader (BioTek) to generate survival curves. Percent survival values were normalized to untreated controls and half-maximal inhibitory concentration/IC₅₀ values were determined by calculating the nonlinear regression using sigmoidal dose-response equation (variable slope). Area under the survival curve/AUSC was calculated using trapezoidal rule and log₂-transformed for further statistical analysis (Mitra et al., 2016). Caspase3/7 activity were evaluated using Caspase-Glo®3/7 Assay kit (Promega) on Synergy 2 Microplate Reader.

### Gene Expression Profiling (GEP)

RNA was isolated from six (6) most Ix-sensitive and six (6) most Ix-resistant cell lines and RNA sequencing was performed on llumina's HiSeq 2000 using 50 bp paired-end protocol with depth of >20 million reads-per-sample. RNA-seq data from CD138-selected plasma cells were generated from MM patients enrolled in an ongoing phase-2 Ixazomib clinical trial at Mayo Clinic (Mayo-Ix; NCT01415882) that enrolled patients with relapsed myeloma, who had less than 6-cycles of prior treatment with a Bz-based regimen and were not refractory to Bz (Kumar et al., 2015).

Gene expression, treatment arm and outcome data on newly diagnosed mycloma patients enrolled in HOVON65/GMMG-HD4 trial (ISRCTN64455289; n=290) were downloaded from Gene expression omnibus/GEO (GSE19784) (Kuiper et al., 2012). The APEX dataset (GSE9782; n=264) consisted of bortezomib-based phase-2 and phase-3 relapsed and/or refractory myeloma clinical trials (The APEX phase-3 trial (039), a companion study (040), the SUMMIT (025) and CREST phase-2 trials (024)) (Mulligan et al., 2007). For both these trials, Affymetrix gene probeset analysis data (U133-Plus2.0 for HIOVON65/GMMG-H-ID4 and HG-U133A/B for APEX) were available. Pre-treatment RNA-seq data, treatment arm and clinical outcome information on CoMMpass (Relating Clinical Outcomes in MM to Personal Assessment of Genetic Profile) trial patients were downloaded from the MMRF (Multiple Myeloma Research Foundation) researcher gateway portal (IA7c release; https://research.themmrf.org). The CoMMpass Trial (NCT0145429), sponsored by MMRF, is a non-registrational, longitudinal study of 1000 newly diagnosed MM patients followed over the course of their disease, up to 8 years (Kowalski et al, 2016).

### Bioinformatics and Statistical Analysis

All statistical analyses were performed using R software environment ver 3.3.1 for statistical computing and graphics, and GraphPad Prism ver 7.0. Spearman rank-order and Pearson Product-Moment correlation/PPMC analyses were performed to compare the PI responses. All tests were two-sided and p<0.05 was considered statistically significant. Gene expression data was pre-processed. log₂-transformed and analyzed using Galaxy and Partek Genomics Suite v6.6 to perform differential expression testing to identify gene expression signatures PI response. Heatmaps were generated using unsupervised hierarchical clustering/HC analysis based on the differentially expressed genes.

Supervised machine learning approaches construct algorithms that learn from training data, build models based on properties of training inputs and thus make learned predictions/decisions on new/test samples (Mitra et al., 2016; Hastie et al., 2009). Random forest, a supervised ensemble machine learning algorithm, was used to establish the top differentially expressed genes as predictive GEP signatures of PI-response (Hastic et al., 2009). GEP data on top Ix-sensitive vs top Ix-resistant HMCLs (n=12) was used as ‘training dataset’ to build random forest classification models (decision trees) and predict PI-resistance of HMCLs (n=44) in the ‘test dataset’—mRNA-seq data obtained from the Keatslab repository (http://www.keatslab.org/data-repository). The average bootstrap prediction error was generated using repeated bootstrapping of the train dataset with a k-fold cross validation (k=100). The cross validation error rate was used to evaluate the accuracy of the method (Mitra et al., 2016; Hastie et al., 2009).

The predictive GEP signature was then applied to the 4 different PI-based MM clinical trials and random survival forest estimation method for right censored data (randomForestSRC), a supervised machine learning decision-tree based algorithm, was used to predict probability of progression/event within the first 3 years for each myeloma patient (Ishwaran et al., 2008). Random survival forest is a random forest-based ensemble class prediction method for right censored survival data such as patient survival data, disease progression data, and drug treatment data that uses a combination of random forest method and the Kaplan-Meier (KM) estimator (Ishwaran et al., 2008) (see methods).

The predicted probability values derived using machine learning approaches (random forest and randomForestSRC) were rank-ordered and the predictions for the top (Q3) and bottom quantiles (Q1) were compared with observed PI-response using Somers' D_{xy} rank correlation. Somers' D_{xy} is a measure of correlation between a variable x and a binary (0-1) variable y, can vary between [−1,1] and is given by the following equation (Somers, 1962):

D_{xy}=2×(c−0.5)

Where, Somers' c is equivalent to the area under the curve (AUC) of the receiver operating characteristics (ROC) corresponding to the predicted values and the binary outcome (0 or 1) of the test data. High positive Somers' D_{xy} rank correlation value indicates high prediction accuracy of any predictive model.

Unsupervised K-means clustering was performed using the algorithm of Hartigan and Wong (1979) to identify clinically important K subgroups based on our PI-response GEP signature such that N/K˜30 (N=total number of subjects in dataset). Kaplan-Meier/KM curves for survival were generated for the extraordinary PI-response (good vs poor) clusters by computing progression-free survival (PFS) over time. The KM survival curves were compared statistically using log-rank test and Cox proportion hazard test (Arubini et al., 2004). Clusters with n<10 were combined for PFS comparisons.

Odds ratios (OR) between observed clinical responses (available for APEX and CoMMpass datasets) vs extraordinary PI-response K-means clusters were computed using binomial logistic regression analysis (logit model) (Hosmer et al., 2013).

### Ingenuity Pathway Analysis

The differentially expressed genes were analyzed using Ingenuity Pathway Analysis/IPA to identify canonical pathways, downstream effects, upstream regulators and causal networks and to perform predictive toxicology analysis using toxicogenomics approaches (IPA-Tox) (Thomas et al., 2010).

### RNA-Sequencing

High quality RNA was extracted from HMCLs using QIAshredder and RNeasy kit (Qiagen). RNA concentration and integrity were analyzed using the Nanodrop-8000 and Agilent 2100 Bioanalyzer and stored at −80° C. RNA integrity number/RIN>8 was considered suitable for RNA-seq analysis. RNA-seq library construction was performed using the Illumina TruSeq RNA sample Preparation kit v2. The libraries were size selected to generate inserts of about 200 bp and RNA sequencing was performed using llumina's HiSeq 2000 next-generation high-throughput sequencing system using 50 bp paired-end protocol with depth of >20 million reads-per-sample. Average quality scores were well above Q30 for all libraries in both R1 and R2. Data was normalized and FPKM values were used in further analysis using a combination of Galaxy data analysis software and Partek Genomics Suite. GEP vs in vitro chemo-sensitivity data was then used to identify gene expression signatures associated with PI response.

### Mayo Clinic Ixazomib Trial

Bone marrow samples were obtained from patients prior to enrollment. RNA extraction was performed on CD138-selected plasma cells using AllPrep DNA/RNA mini kit (Qiagen). 100 ng high-quality RNA were used for RNA-seq library construction using the Illumina TruSeq RNA sample Preparation kit v2 (Illumina Inc., San Diego, Calif., USA). The libraries were size selected to generate inserts of about 200 bp and RNA sequencing was performed using llumina's HiSeq 2000 next-generation high-throughput sequencing system using 109 bp paired-end protocol with depth of about 100 million reads per sample (Illumina Inc., San Diego, Calif., USA).

### RNA-Seq Data Processing

Gene expression data was pre-processed using Galaxy, an open source, web-based platform that provides tools necessary to create and execute RNA-seq analysis. Briefly, an RNA-seq data analysis pipeline was developed using Galaxy that performs quality control (QC) check on the RNA-seq raw reads using FastQC tool, trims the reads to remove base positions that have a low median (or bottom quartile) score and then uses Tophat2 tool to map processed RNA-seq reads to the hg19 human genome build. Estimated insert sizes were derived using Picard's CollectlnsertSizeMetrics tool on this initial tophat2 run which was used to calculate mean inner distance between mate pairs (Mean=estimated_insert-size−2*read_length). Tophat2 was then re-run using correct mean value and finally Cufflinks tool was used on these datasets to assemble the reads into transcripts. Prior to analysis, the processed transcripts were filtered using the following criteria: genes with variance=0. FPKM<1 and mean FPKM<5 were removed.

Random Forest Random forest is a decision tree-based ensemble machine learning method that constructs multiple decision trees by randomly splitting the data from training dataset in its feature space and predicts the modal class in classification models and the mean prediction in regression models (Breiman, 2001). In the analysis, random forest-based classification model approach that used Pi-sensitive vs PI-resistance information of the training dataset (6 most Ix-sensitive vs 6 most Ix-resistant HMCLs) was used to predict PI-resistance in the test dataset of HMCLs (mRNA-seq data obtained from the Keatslab repository, http://www.keatslab.org/data-repository) using the 42 gene GEP signature of PI-response. The average bootstrap prediction error was generated using repeated bootstrapping of the train dataset with a k-fold cross validation with k=100. The cross validation error rate was used to evaluate the accuracy of the method.

### Random Survival Forest

Random survival forest is a random forest-based ensemble class prediction method for right censored survival data such as patient survival data, disease progression data, and drug treatment data (Ishwaran et al., 2008). Random survival forest estimation for right censored data is performed based on a combination of the random forest method (ensemble of decision trees) and the Kaplan-Meier (KM) estimator for survival data. The tree based survival function is computed by using the Kaplan Meier (KM) estimator for a decision tree's terminal node. To be precise, if Yₕ⁽ⁿ⁾(t) be the number of individuals in terminal node h of a random survival forest at time t, i.e., the number of individuals in a terminal node who are at risk, but have neither experienced an event nor have been censored, and if Nₕ⁽ⁿ⁾(t) be the number of events in the interval (0, t] for all cases in h, then the KM estimator for cases within h is given by the following equation.

$${\hat{S}}_{h} ( t ) = {\prod }_{S \leq t} ( 1 - \frac{{dN}_{h}^{( n )} ( s )}{{Y}_{h}^{( n )} ( t )} )$$

The survival function for individual i is the average of B bootstrapped KM estimator for the terminal node where individual i belongs and is given by the equation.

$$\hat{S} ( t \vert {x}_{i} ) = \frac{1}{B} {\sum }_{b = 1}^{B} {\hat{S}}_{b} ( t \vert {x}_{i} ) = \frac{1}{B} {\sum }_{b = 1}^{B} {\sum }_{h} I ( {x}_{i} ∈ h ) \hat{{S}_{h}} ( t )$$

The random survival forest estimation was done using R package randomForestSRC.

### Somers' D_{xy} Rank Correlation

The evaluation of random forest and random survival forest prediction methods was performed using Somers' D_{xy} rank correlation between predicted percentage values on test data and the binary outcome (chemosensitivity/progression index) of the test data.

Somers' D_{xy} is a measure of correlation between a variable x and a binary (0-1) variable y and is given by the following equation (Somers, 1962):

D_{xy}=2×(c−0.5)

Where, c is equivalent to the area under the curve (AUC) of the receiver operating characteristics (ROC) corresponding to the predicted values and the binary outcome (0 or 1) of the test data. The Somers' D_{xy} indicates the incremental predictive accuracy beyond random assignment and can vary between [−1,1]. A high positive Somers' D_{xy} rank correlation value indicates high prediction accuracy of any predictive model.

The Somers' D_{xy} rank correlation of predicted values from the random survival forest model was computed using the somers2 function from the package Hmnisc in R (https://cran.r-project.org/web/packagcs/Hmisc/Hmisc.pdf).

### Results

### Wide Variability in Response to PI-Treatment

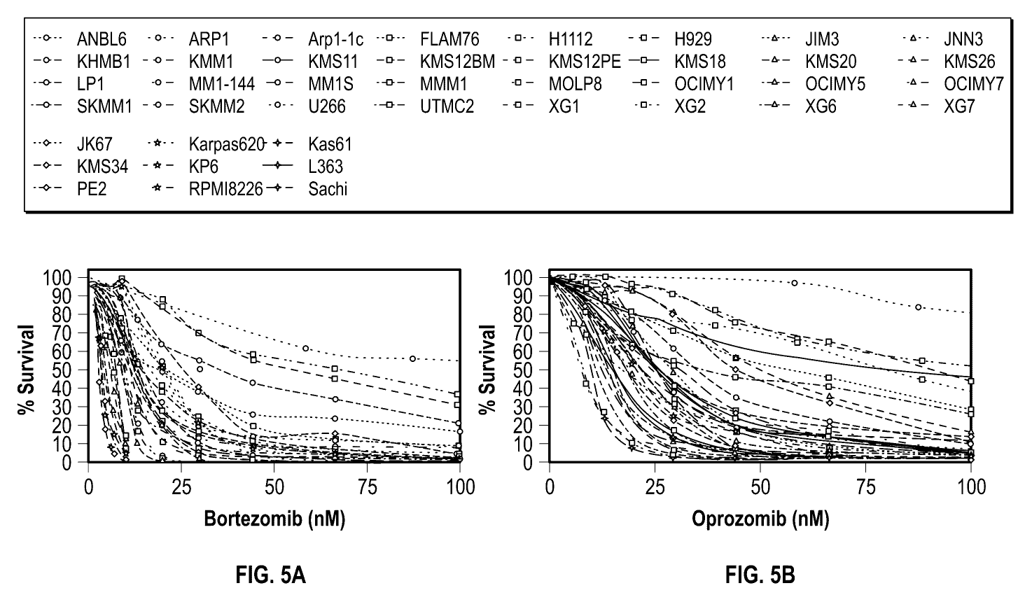

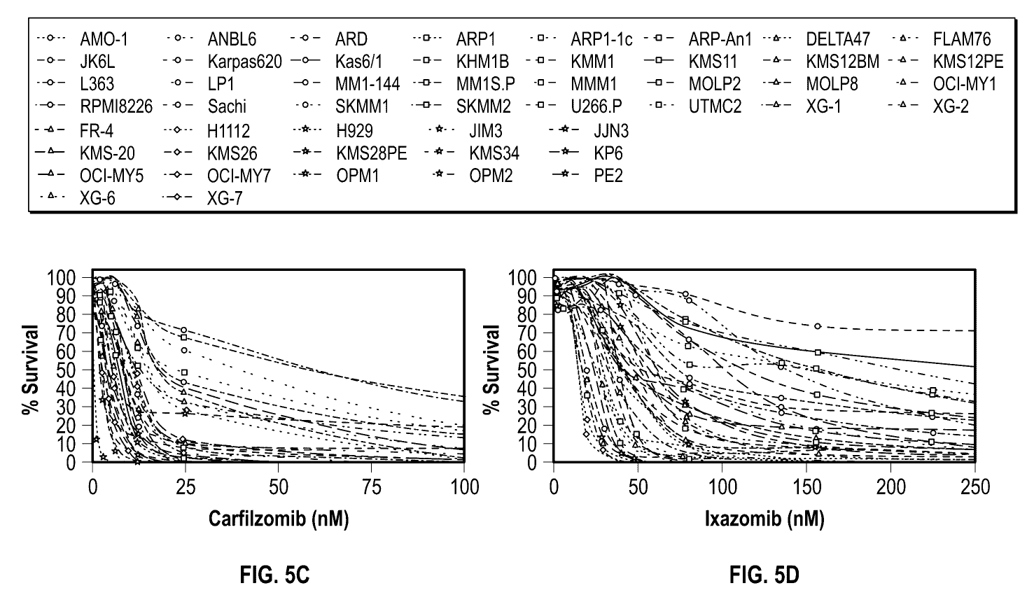

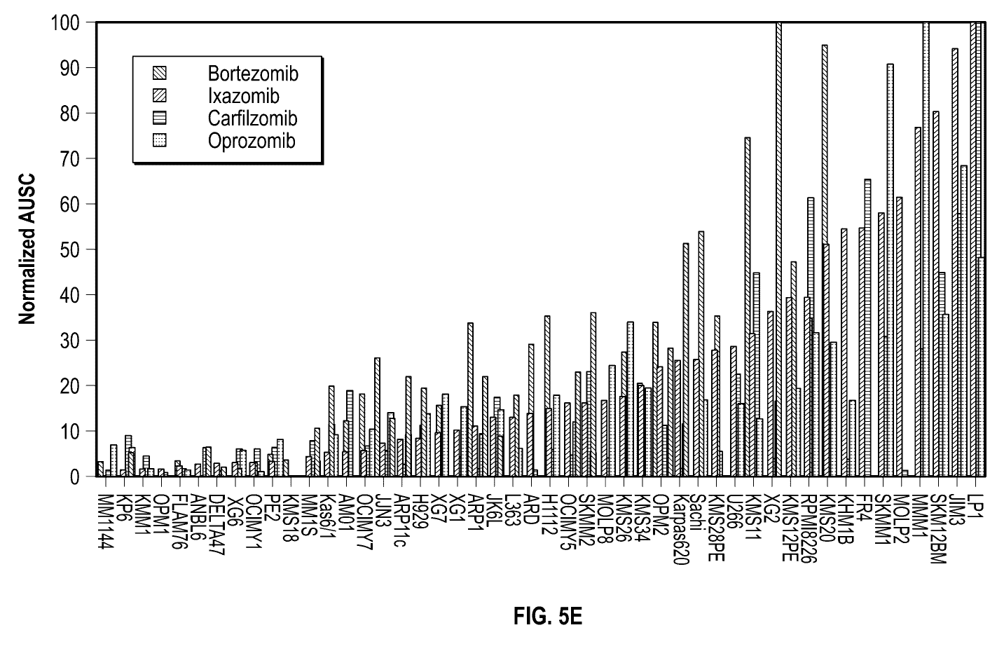

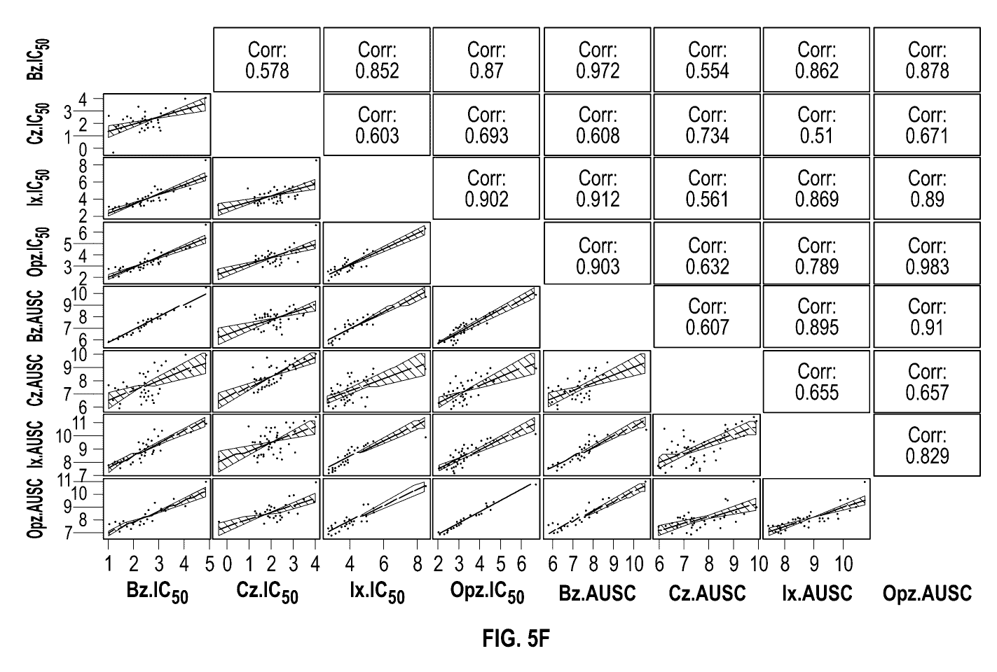

To replicate and characterize the inter-individual variation in therapeutic response, the in vitro PI-sensitivity of a large panel of human myeloma cell lines (HMCLs) was assessed. Results of the cytotoxicity assays in 50 HMCLs showed a wide range of variability in response to treatment with the four PIs (Bz, Cz, Ix and Opz) identifying some lines that are highly sensitive and some lines relatively refractory to PIs, as represented by the IC₅₀ and Area Under the Survival Curve (AUSC) plots (FIGS. 5A-D). In some cases the AUSC provides a comparative measure of response through the higher test doses, especially if an IC₅₀ value was not achieved. The summary table (Table 1B) depicts the extensive variation in response to each of the PIs among the panel of HMCLs. The caspase 3/7 activity data corroborated with cytotoxicity data (data not shown). FIG. 5E shows a correlation matrix representing comparative analysis of IC₅₀ values of four PIs across the HMCLs. Statistically significant (adjusted p-values<0.001; Holm's method) positive correlation was observed between IC₅₀ values across the four PIs. Thus, in general, sensitivity was highly correlated among all four PIs. However, some examples of HMCLs that were highly sensitive to one PI, and comparably less-sensitive to other PIs were found. This demonstrates tumor heterogeneity even in response to inhibitors of the same class, and further demonstrates some tumors refractory to one PI may still respond to another.

TABLE 1B
Numerical summaries of chemo-sensitivity parameters in HMCLs

| | Mean | Minimum | Median | Maximum |
|---|---|---|---|---|
| Proteasome inhibitor | (nM) | (nM) | (nM) | (nM) |
| Bortezomib_ IC₅₀ | 17.1 | 2.8 | 11.7 | 124.3 |
| Carfilzomib_ IC₅₀ | 10.9 | 0.7 | 7.1 | 55.3 |
| Ixazomib_ IC₅₀ | 155.3 | 15.1 | 42.1 | 4757.9 |
| Oprozomib_ IC₅₀ | 45.8 | 7.6 | 23.7 | 776.0 |
| Bortezomib_ AUSC | 2700.1 | 319.6 | 1524.0 | 38974.0 |
| Carfilzomib_ AUSC | 3503.4 | 375.5 | 1448.5 | 19097.0 |
| Ixazomib_ AUSC | 10030.0 | 1702.0 | 6456.0 | 46494.0 |
| Oprozomib_ AUSC | 5017.0 | 1050.0 | 2885.0 | 59917.0 |

### Deriving a GEP-Based Signature Profile of PI-Response

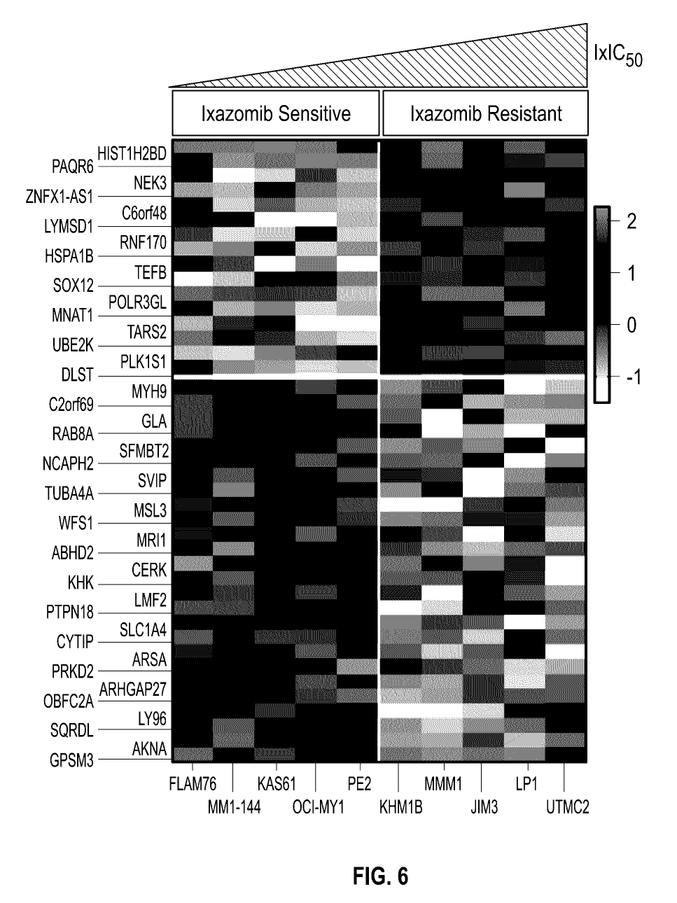

A differential gene expression analysis was performed between 5 (top-10%) most Ix-sensitive and 5 (bottom-10%) most Ix-resistant cell lines. Notably, these cell lines showed the same relative sensitivity and resistance to all four proteasome inhibitors. RNA-seq data was pre-filtered, normalized and used for further analysis. Differential gene expression analysis was performed using the remaining 7682 genes to identify GEP signatures that distinguish the highly sensitive from the highly resistant HMCLs. Results showed 506 genes differed significantly between the sensitive and the resistant groups (p<0.05; fold-difference≠1). 141 genes showed |fold-difference|>2 and p<0.05, while 42 genes, listed in Table 2, had p<0.01 (|fold-difference|>2) (FIG. 6). Subsequent analyses used the more stringent 42-gene list.

TABLE 2
List of genes most significantly associated with PI resistance (|Fold-
difference| > 2; p < 0.01). Differential gene expression
analysis was performed to compare gene expression profiles of 5 (Top
10%) most Ix-sensitive and 5 (Bottom 10%) most Ix-resistant cell lines.
These 42 genes were used as GEP signature of PI resistance to stratify
PI response in test datasets (in vitro and among patients).

| | | | Fold-difference | |
|---|---|---|---|---|
| | | | (Sensitive vs. | Fold-difference |
| # | Gene ID | p-value | Resistant) | (Description) |
| 1 | SLC1A4 | 0.00004 | 2.695 | Sensitive up vs Resistant |
| 2 | NEK3 | 0.00006 | −2.544 | Sensitive down vs |
| | | | | Resistant |
| 3 | GLA | 0.00007 | 2.073 | Sensitive up vs Resistant |
| 4 | AKNA | 0.00020 | 4.043 | Sensitive up vs Resistant |
| 5 | ARHGAP27 | 0.00035 | 3.599 | Sensitive up vs Resistant |
| 6 | LY96 | 0.00045 | 3.796 | Sensitive up vs Resistant |
| 7 | DLST | 0.00070 | −2.001 | Sensitive down vs |
| | | | | Resistant |
| 8 | MSL3 | 0.00132 | 2.237 | Sensitive up vs Resistant |
| 9 | SQRDL | 0.00134 | 3.906 | Sensitive up vs Resistant |
| 10 | NCAPH2 | 0.00206 | 2.129 | Sensitive up vs Resistant |
| 11 | PLK1S1 | 0.00262 | −2.035 | Sensitive down vs |
| | | | | Resistant |
| 12 | MRI1 | 0.00284 | 2.448 | Sensitive up vs Resistant |
| 13 | TARS2 | 0.00294 | −2.083 | Sensitive down vs |
| | | | | Resistant |
| 14 | OBFC2A | 0.00307 | 3.756 | Sensitive up vs Resistant |
| 15 | RAB8A | 0.00319 | 2.097 | Sensitive up vs Resistant |
| 16 | ABHD2 | 0.00363 | 2.475 | Sensitive up vs Resistant |
| 17 | LMF2 | 0.00364 | 2.558 | Sensitive up vs Resistant |
| 18 | C6orf48 | 0.00367 | −2.462 | Sensitive down vs |
| | | | | Resistant |
| 19 | TUBA4A | 0.00400 | 2.189 | Sensitive up vs Resistant |
| 20 | HSPA1B | 0.00467 | −2.335 | Sensitive down vs |
| | | | | Resistant |
| 21 | TFEB | 0.00471 | −2.302 | Sensitive down vs |
| | | | | Resistant |
| 22 | RNF170 | 0.00504 | −2.34 | Sensitive down vs |
| | | | | Resistant |
| 23 | SOX12 | 0.00569 | −2.281 | Sensitive down vs |
| | | | | Resistant |
| 24 | ZNFX1-AS1 | 0.00604 | −2.482 | Sensitive down vs |
| | | | | Resistant |
| 25 | C2orf69 | 0.00622 | 2.038 | Sensitive up vs Resistant |
| 26 | PTPN18 | 0.00634 | 2.635 | Sensitive up vs Resistant |
| 27 | PRKD2 | 0.00641 | 3.5 | Sensitive up vs Resistant |
| 28 | KHK | 0.00662 | 2.484 | Sensitive up vs Resistant |
| 29 | PAQR6 | 0.00710 | −3.645 | Sensitive down vs |
| | | | | Resistant |
| 30 | HIST1H2BD | 0.00763 | −4.514 | Sensitive down vs |
| | | | | Resistant |
| 31 | CERK | 0.00776 | 2.481 | Sensitive up vs Resistant |
| 32 | UBE2K | 0.00806 | −2.048 | Sensitive down vs |
| | | | | Resistant |
| 33 | LYSMD1 | 0.00814 | −2.379 | Sensitive down vs |
| | | | | Resistant |
| 34 | GPSM3 | 0.00832 | 5.309 | Sensitive up vs Resistant |
| 35 | MNAT1 | 0.00906 | −2.144 | Sensitive down vs |
| | | | | Resistant |
| 36 | WFS1 | 0.00911 | 2.397 | Sensitive up vs Resistant |
| 37 | MYH9 | 0.00923 | 2.011 | Sensitive up vs Resistant |
| 38 | CYTIP | 0.00925 | 2.855 | Sensitive up vs Resistant |
| 39 | SVIP | 0.00928 | 2.148 | Sensitive up vs Resistant |
| 40 | ARSA | 0.00931 | 3.3 | Sensitive up vs Resistant |
| 41 | SFMBT2 | 0.00947 | 2.111 | Sensitive up vs Resistant |
| 42 | POLR3GL | 0.00958 | −2.251 | Sensitive down vs |
| | | | | Resistant |

### GEP Signature of PI Response is Predictive of In Vitro PI-Chemosensitivity in HMCLs and Progression in MM Clinical Trials

Using random forest algorithm for classification, a probability score for PI resistance was computed based on the 42-gene signature in a new ‘test’ dataset of mRNA-seq data on HMCLs (n=44) obtained from the Keatslab data repository (http://www.keatslab.org/data-repository. Briefly, the model was ‘trained’ using 6 most Ix-sensitive and 6 most Ix-resistant cell lines to construct classification trees which then predicted PI-resistance of HMCLs in the test dataset. The predicted probabilities of PI-resistance were rank-ordered and Somers' D_{xy} rank correlation analysis was performed between the top quantile/Q3 and bottom quantile/Q1 resistance probability values vs observed PI-chemosensitivity as a binary outcome (sensitive=0 vs resistance=1).

Results revealed high positive Somers D_{xy} rank correlation for the 4 PI drug cytotoxicity parameters (IC₅₀ and AUSC) (Table 3), indicating that the 42-gene GEP signature validated quite well in an independent dataset of HMCLs. Since the classification model was generated using HMCLs with top-6+bottom-6 Ix IC₅₀ values as training dataset, the test dataset for Ix IC₅₀ prediction included the remaining 32 cells lines only. Among these, Somers' c value for correlation between observed Ix-IC₅₀ versus response probabilities of the top 6 predicted Ix-resistant cell lines was 0.667 while was 1.0 for the top 6 predicted Ix-sensitive lines. Somers' c for the combined set of 12 predicted sensitive+resistant HMCLs was 0.743. Thus, the independent cell line test set showed very good correlation with the signature derived predictor.

TABLE 3
Summary of correlation between predicted probabilities of PI-
resistance vs observed PI cytotoxicity values. Random forest
classification model was generated using HMCLs with top-6 + bottom-6
Ix-IC₅₀ values as training dataset. Predicted probability values
of HMCLs in the test dataset were rank-ordered and Somers'
Dxy rank correlation analysis was performed between the top quantile/Q3
and bottom quantile/Q1 resistance probability values observed
PI-chemosensitivity as a binary outcome (sensitive = 0 vs
resistance = 1). Spearman rank-ordered correlation was performed
in cell lines representing Q3 and Q1 probabilities of resistance
and corresponding cytotoxicity values,

| | Somers' c | | | Spearman's rho | |
|---|---|---|---|---|---|
| | c_{Q3} | c_{Q1} | C_{Q3+Q1} | Spearman_{Q3+Q1} | P |
| Bz_IC50 | 0.643 | 0.786 | 0.852 | 0.748 | 0.00036 |
| Cz_IC50 | 0.714 | 0.524 | 0.750 | 0.563 | 0.00981 |
| Opz_IC50 | 0.667 | 0.944 | 0.802 | 0.626 | 0.00548 |
| Bz_AUSC | 0.643 | 0.786 | 0.852 | 0.736 | 0.00050 |
| Cz_AUSC | 0.595 | 0.667 | 0.712 | 0.601 | 0.00507 |
| Ix_AUSC | 0.857 | 0.889 | 0.927 | 0.765 | 0.00009 |
| Opz_AUSC | 0.667 | 0.786 | 0.813 | 0.630 | 0.00509 |

While the training set showed good prediction capabilities in an independent set of HMCLs, it was of interest to determine if it stratifies patient outcomes in multiple clinical trials. Four different PI-treating trials were examined. Microarray gene expression data from clinical trials was mean-centered and scaled prior to analysis. The standardized transcriptomic profiling data from APEX trial were 3 used as a training dataset to perform Random survival forest analysis to predict the percent probability of a progression/event within 3 years in the HOVON-GMMG-HD4, CoMMpass and Mayo-Ix clinical trials (test datasets). Somers' D_{xy} rank correlation analysis between the top and bottom quantiles of predicted percentage values from random survival forest model on test data and the progression index of the test datasets showed consistent positive values for the HOVON-GMMG-HD4, CoMMpass and Mayo-Ix clinical trials revealing high prediction accuracy of the random survival forest-based prediction model (Ishwaran et al., 2008; Somers, 1962) (Table 4). Somers' c for the training dataset (APEX) was =0.852.

TABLE 4
Summary of Somers' Dxy rank correlation analysis between
predicted probability values of progression (derived from random
survival forest model) and the progression index of MM patients
from PI-based clinical trials (test datasets). Transcriptomic
profiling data from APEX trials was used as training dataset.

Somers' c

| | c_{Q3} | c_{Q1} | C_{Q3+Q1} |
|---|---|---|---|
| HOVON-GMMG-HD4 (PAD Arm) | 0.596 | 0.599 | 0.561 |
| CoMMpass | 0.705 | 0.469 | 0.595 |
| Mayo-Ix | 0.500 | 0.833 | 0.680 |
| APEX-Dex Arm | 0.365 | 0.467 | 0.431 |

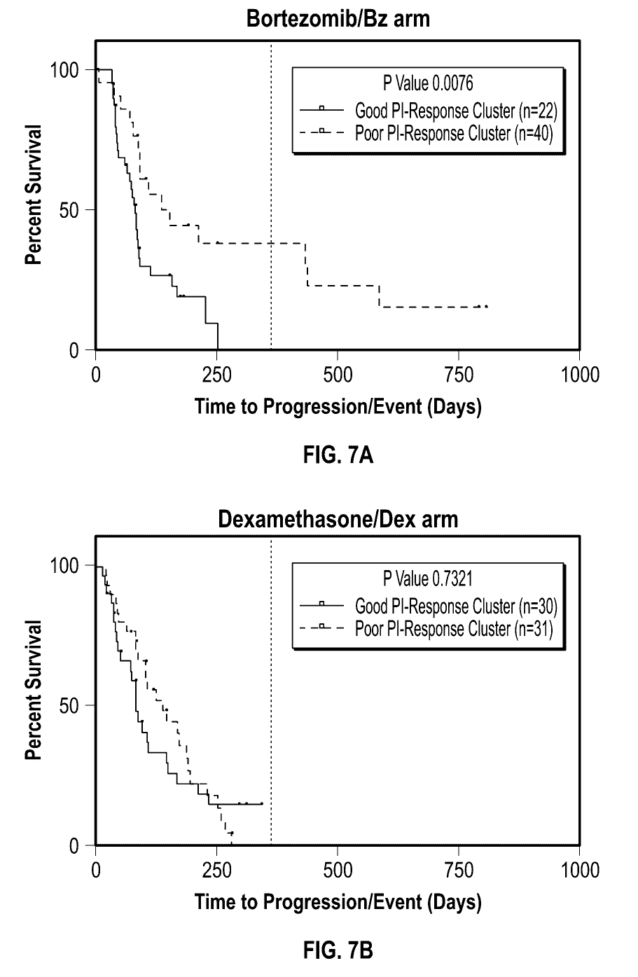

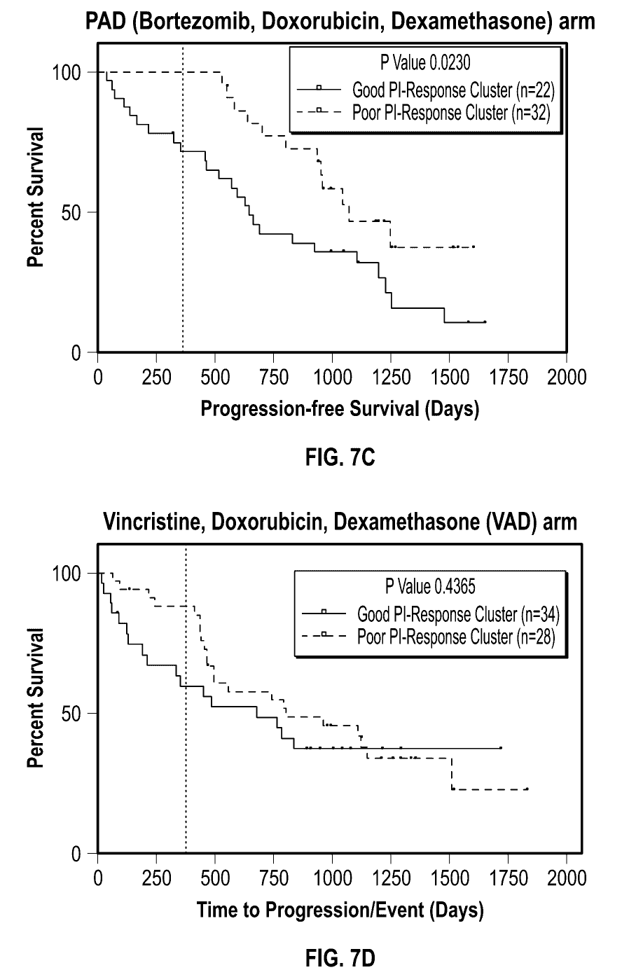

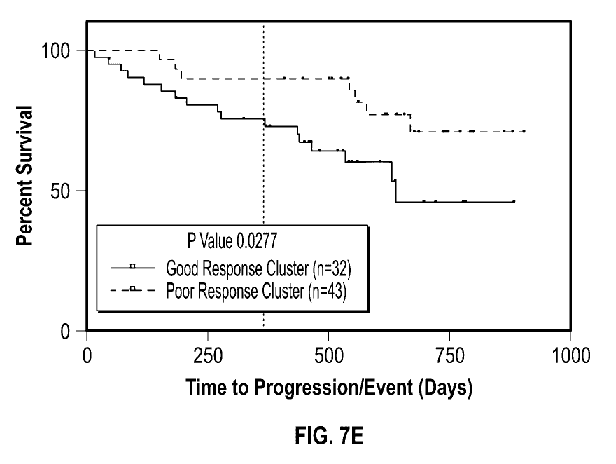

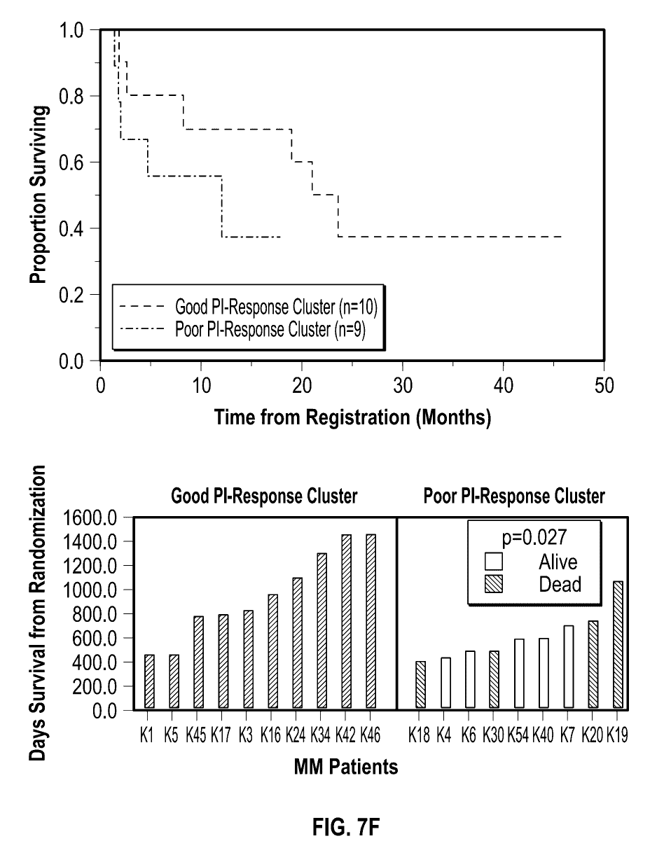

Since the signature's drug response performance was of interest, progression was chosen as one measure of response/non-response as well as associations that distinguish clinical definition of response, complete response (CR) versus non-response (NR) (Rajkumar et al., 2011). K-means clustering was performed on 188 MM patients from the Bz treatment arm of the APEX dataset (Mulligan et al., 2007), to partition the samples into clusters/subgroups based on the expression of the 42 genes comprising the GEP signature of PI response. Results show significant differences in PFS between the signature-predicted poor PI-response versus good PI-response groups (Hazard ratio (HR)=2.346; P=0.0076) (FIG. 7A). Conversely, no difference in PFS is observed between the K-means clusters when the 42-gene model was applied to the expression data of 76 patients in the dexamethasone arm of APEX phase-3 trial that compared single-agent Bz with high-dose dexamethasone (HR=1.1; P=0.732;) (FIG. 7A), showing the GEP signature is drug-specific. Concurrently, statistically significant association is observed between the K-means clusters and clinical response in the Bz arm (OR_{responder (R) vs non-responder (NR)}=5.81³; 95% CI=1.833-20.007; P_{OR}=0.0036) but not in the Dex arm (OR_{RvsNR}=2.139; 95% CI=0.753−6.326; P_{OR}=0.158) of the APEX dataset.

When applied to the gene expression data from HOVON-GMMG-HD4 clinical trial (n=290) that implemented a Bz-based drug regimen (Kuiper et al., 2012), the 42-gene signature shows statistically significant differences in PFS among K-means clusters representing good versus poor PI-response in the Bz-treated PAD (bortezomib, doxorubicin, and dexamethasone) arm (HR=2.161; P=0.024), while no difference in PFS is observed in the vincristine, doxorubicin, and dexamethasone (VAD) arm (HR=1.282; P=0.437) (FIG. 7B). What is particularly striking is that within the first 1.5, years the predicted good response group in the PI-containing therapy had no progression events, while 35% of the predicted poor response group had progression events.

Clinical data was available on 765 patients from the CoMMpass study. 253 patients remained after applying the following filtering criteria: Inclusion criteria—Bz used as first-line of therapy, alone or in combinations; Exclusion criteria—i) Bortezomib as induction, maintenance or consolidation therapy; ii) other or unknown treatment regimen. 128 of these had RNA-seq and PFS data available. K-Means clustering based on the expression of the 42 genes distinguishes between good vs poor PFS subgroups of myeloma patients (HR=2.556; P=0.0277) (FIG. 7C). In addition, the K-Means clusters are also found associated with clinical response (OR=5.20; 95% CI=1.22 36.077; P_{OR}=0.0453).

Finally, to further validate the 42-gene PI-response prediction signature, the algorithm was applied to an ongoing clinical trial at Mayo Clinic that uses Ix-containing drug regimen. Interestingly, even with small numbers of patients available, the GEP-based PI-response classifier distinguishes between the top (medium-PFS=22.42 months) versus bottom responders (medium-PFS=12.155 months) in this Ix trial (risk-ratio=2.5) (FIG. 7D). Notably, all 10 patients in the good performance group were alive at the latest point of the interim analysis, while 4 out of 9 patients in the poor PI-response cluster died (inset of FIG. 7D).

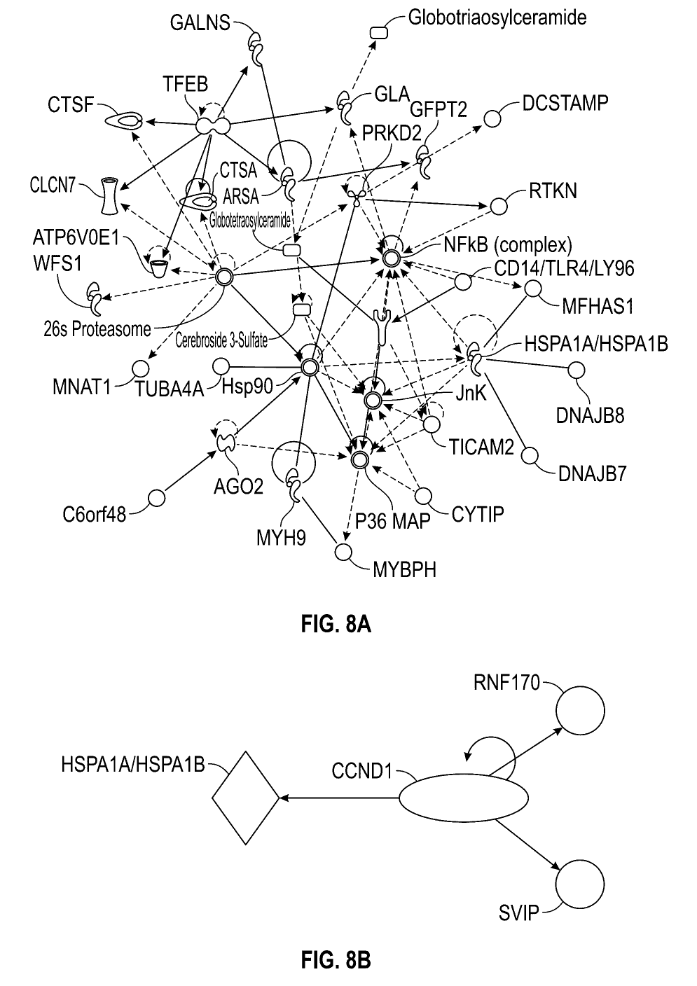

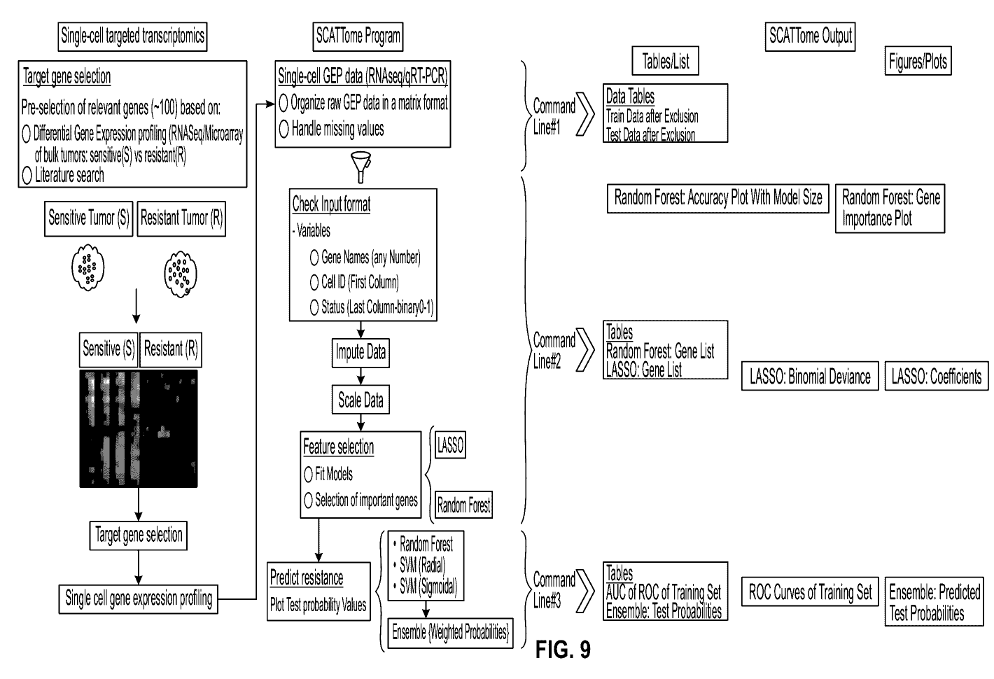

Genes in the 42-gene GEP model were then analyzed using Ingenuity Pathway Analysis (IPA) to assign them to different functional networks. 12 out of the 42 genes overlapped with the top IPA network obtained from the analysis of direct and indirect relationships (score=26), as represented in FIG. 8A. Genes/molecules in this network include the 26s proteasome, Hsp90 and NFκB complex. Further, when the IPA upstream regulator analysis was used. TFEB and CCND1 were identified as the top hits/factors that may control the genes and pathways highlighted by network analysis (FIG. 8B).

### DISCUSSION

Resistance to chemotherapy in cancers is a major limitation in achieving A complete and sustained therapeutic effect, and may in fact lead to unwanted exposure to ineffective anti-tumor agents thereby increasing the risk of negative side-effects (Marin et al., 2012). Multiple Myeloma is an incurable disease with a considerably low median survival of around 7 years despite significant improvement in treatment strategies over the last 12 years (Richardson et al., 2007; Fonesca et al., 2008; Vangsted et al., 2012; Munshi et al., 2013; Kuehl et al., 2012). Differential gene expression profiling studies have provided GEP signatures as a useful prognostic indicator of low versus high risk MM; however, none of these gene signatures were treatment specific (Moreaux et al., 2011; Kuiper et al., 2012; Zhan et al., 2008). Furthermore, no study thus far has made use of the vast array of HMCLs as model system to generate drug chemosensitivity profile as a representation of the response variation in patient subtypes that may be used to derive a PI-specific GEP signature predictive of resistance and treatment outcomes.

In the current study, using a panel of 50 HMCLs, an in vitro chemo-sensitivity profile to four PI treatments was developed. The focus was on common features that distinguish the most and least responsive HMCLs, noting that these responses are very similar across all four PIs. Several outliers (HMCLs that were highly resistant to one PI and highly sensitive to another) were noted. Interestingly, several cell lines with intermediate response to Bz or Ix were found highly responsive to Cz-treatment. But in general the ranked correlation with response was very similar among all four PIs, particularly those lines that were collectively the most responsive and least responsive to all four PIs. Further characterization of the outliers may reveal important features that better direct which PI is most effective.

Using transcriptomic profiling of PI-sensitive versus resistant HMCLs followed by machine-learning based response prediction, a gene expression signature that distinguishes strong sensitivity versus strong resistance to the four PIs when used in MM was demonstrated. Importantly, this signature was not applied across the full range of responses, but was very effective in identifying extraordinary (good versus poor) response. The 42-gene signature of innate PI-response is different from a signature of acquired PI-response that was generated by comparing GEP between PI-sensitive and subsequent resistant outgrowths of Bz-resistant MM cell lines (unpublished data).

Despite the small numbers of cell lines used to generate the 42-gene signature, when the signature was applied to clinical trials on MM patients undergoing PI-based therapies, the GEP signatures were remarkably correlated with PI response/resistance, could predict early or late progression events, and efficiently clustered patient responses into favorable (R or CR) vs unfavorable (NR) outcome subgroups. Notably, the GEP signature did not stratify response to dexamethasone in the APEX trial, suggesting PI-treatment specificity, not simply high versus low risk tumor biology irrespective of drug treatment regimen as has been previously reported (Zhan et al., 2008).

Several genes included in the GEP signature have been previously implicated with prognosis and disease progression in myeloma. For example, the cell cycle regulator CCND1 (cyclin-D1) has been reported as a strong prognostic predictor in multiple studies (Moreaux et al., 2011; Mitra et al., 2016). HSPA1B is an NRF2-mediated oxidative stress response gene which was also found significant (p<0.001; |fold-change|>2) in an earlier study that compared differential responsiveness to Bz in vitro (Stressman et al., 2013). Abnormal levels of serum bone Gla protein have been shown to play important role in the inhibition of bone formation in the occurrence of bone lesions in MM which may have prognostic implications (Bataille et al., et al., 1990). Wolfram syndrome 1/WFS1 encodes an endoplasmic reticulum/ER transmembrane protein that plays a role in regulation of ER stress signaling and in the pathogenesis of diseases involving chronic, unresolvable ER stress (Fonseca et al., 2010). Further, WFS1 is a downstream transcriptional target of the gene X-box binding protein/XBP1, a bZIP transcription factor that regulates UPR and ER stress-induced apoptosis (Fonseca et al., 2010; Gamella et al., 2014). Higher expression of XBP1 pathway genes is associated with better outcome/response in PI-treated MM patients while loss of XBP1 has been shown to induce PI resistance (Gambella et al., 2014; Leung-Hagesteijn et al., 2013). Interestingly, we found that the unspliced transcript of XBP1 (Xbp1u) had about 1.5 times lower expression in Ix-resistant cell lines when compared to Ix-sensitive cell lines. The transcription factor EB/TFEB is a master gene for lysosomal biogenesis that drives expression of a large subset of autophagy genes leading to activation of autophagic-cell death of myeloma cells owing to metabolic cell stress (Cea et al., 2013). RNF170 encodes an endoplasmic reticulum membrane ubiquitin ligase that mediates ubiquitination and degradation and plays key role in cell signaling (Lu et al., 2011). Given previous reports on the association of these genes to myeloma biology and outcomes, their collective inclusion in unsupervised clustering further validates their role in PI response. While individually associated with MM biology, the GEP signature suggests a combination of effectors influence response to PIs.

Earlier, it was demonstrated that gene expression signatures may be used to identify secondary therapies in PI-resistant MM using in silico predictions that were confirmed in vitro (Stressman et al., 2013; Stressman et al., 2014). Therefore, the present serves as resource to use machine learning-based approaches for the personalized prediction of chemo-resistance and to eventually identify signatures of drug combination regimens that may effectively reverse drug resistance by predicting drugs for various subpopulations/subclones of tumors based on pharmacogenomic signature profiles.

### REFERENCES

Adams et al., Cancer Res., 59:2615 (1999).
Altman, Practical Statistics for Medical Research, 1 (1991).
Bataille et al., Cancer. 66:167 (1990).
Boylan et al., Cancer Res., 67:4069 (2007).
Bradley, Pattern Recognit., 30:1145 (1997).
Breiman et al., Mach. Learning, 45:5 (2001).
Cancer Genome Atlas Research Network, Nature, 455:1061 (2008).
Cea et al., Autophagy, 9:410 (2013).
Costello et al., Nat. Biotechnol., 32:1202 (2014).
Fonseca et al., J. Clin. Invest., 120:744 (2010).
Fonseca et al., Leukemia, 23:2210 (2009).
Gambella et al., Haematologica. 99:e14 (2014).
Garnett et al., Nature, 483:570 (2012).
Hanley et al., Radiology, 143:29 (1982).
Hartigan et al., J. Royal Stat. Soc., Series C. 28:100 (1979)
Hastie et al., Elements of Statistical Learning: Data Mining, Inference and Prediction, 745 (2009).
Hosmer et al., Applied Logistic Regression. 398 (2013).
Ishwaran et al., Annals. Applied Statistics, 2:841 (2008).
Kortuem et al., Carfilzomib. Blood. 121:893 (2013).
Kowalski et al., Nucleic Acids Res., 44:e69 (2016).
Kuehl et al., J. Clin. Invest., 122:3456 (2012).
Kuiper et al., Leukemia, 26:2406 (2012).
Kumar et al., Blood Cancer J., 5:e338 (2015).
Kumar et al., Blood. 2008; 112:2177 (2008).
Leung-Hagesteijn et al., Cancer Cell, 24:289 (2013).
Lightcap et al., Clin. Chem., 46:673 (2000).
Lu et al., J. Biol. Chem., 286:24426 (2011).
Marin et al., Curr. Cancer Drug Targets. 12:402 (2012).
Marubini et al., Analyzing Survival Data from Clinical Trials and Observational Studies. 15 (2004).
Mitra et al., Leukemia, 30:1094 (2016).
Mitsiades et al., J. Clin. Oncol., 29:1916 (2011).
Moreau et al., Blood, 120:947 (2012).
Moreaux et al., Haematologica, 96:574 (2011).
Mulligan et al., Blood, 109:3177 (2007).
Munshi et al., Clin. Cancer Res., 19:3337 (2013).
Rajkumar et al., Blood, 117:4691 (2011).
Rajkumar et al., Lancet Oncol., 15:e538 (2014).
Rajkumar. Am. J. Hematol., 88:226 (2013).
Rajkumar. Am. J. Hematol., 91:90 (2016).
Richardson et al., Blood, : (2015).
Richardson et al., Br. J. Haematol., 137:429 (2007).
Somers, Am. Sociol. Rev., pp. 799-811 (1962).
Stessman et al., Leukemia, 28:2263 (2014).
Stessman et al., Mol. Cancer Ther., 12:1140 (2013).
Thomas et al., Hum. Genomics, 4:353 (2010).
Tibshirani, J. Royal Stat. Soc., 73:273 (2011).
Vangsted et al., Eur. J. Haematol., 88:93 (2012).
Zhan et al., Blood, 111:968 (2008).

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

What is claimed is:

1. A method to predict proteasome inhibitor response or resistance in a multiple myeloma patient, comprising: providing a profile from a gene expression panel from myeloma cells of the patient, wherein the profile includes gene expression from a plurality of genes that are upregulated or downregulated in a plurality of proteasome inhibitor sensitive multiple myeloma cell lines relative to corresponding gene expression in proteasome inhibitor resistant multiple myeloma cell lines; and administering one or more selected proteasome inhibitors to the patient if the profile of the patient has a plurality of genes upregulated or downregulated that are upregulated or downregulated in the plurality of proteasome inhibitor sensitive multiple myeloma cell lines or administering a non-proteasome inhibitor anti-cancer drug or a combination of anti-cancer drugs to the patient if the profile of the patient has a plurality of the genes that are upregulated or downregulated in the proteasome inhibitor resistant multiple myeloma cell lines relative to the plurality of in the proteasome inhibitor sensitive multiple myeloma cell lines.

2. The method of claim 1 wherein one or more of NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3GL, is downregulated and one or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2 is upregulated, or any combination thereof, in the profile of the patient relative to expression in the proteasome inhibitor resistant multiple myeloma cell lines.

3. The method of claim 1 wherein one or more of NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3GL is upregulated and one or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2 is downregulated, or any combination thereof, in the profile of the patient relative to expression in the proteasome inhibitor resistant multiple myeloma cell lines.

4. The method of claim 1 wherein one or more of NEK3, DLST, PLK1S1, TARS2, C6orf48, HSPA1B, TFEB, RNF170, SOX12, ZNFX1-AS, PAQR6, HIST1H2BD, UBE2K, LYSMD1, MNAT1, or POLR3GL, or any combination thereof, is downregulated in the profile of the patient relative to expression in the proteasome inhibitor resistant multiple myeloma cell lines.

5. The method of claim 1 wherein one or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2, or any combination thereof, is upregulated in the profile of the patient relative to expression in the proteasome inhibitor resistant multiple myeloma cell lines.

6. The method of claim 1 wherein one or more of NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3GL, or any combination thereof, is upregulated in the profile of the patient relative to expression in the proteasome inhibitor sensitive multiple myeloma cell lines.

7. The method of claim 1 wherein one or more of SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2, or any combination thereof, is downregulated in the profile of the patient relative to expression in the proteasome inhibitor sensitive multiple myeloma cell lines.

8. The method of claim 1 wherein the plurality of genes includes NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, or POLR3GL, or any combination thereof.

9. The method of claim 1 wherein the plurality of genes includes SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2, or any combination thereof.

10. The method of claim 1 wherein the plurality of genes includes two or more of HIST1H2BD, PAQR6, NEK3, ZNFX1-AS1, C6orf48, LYSMD1, RNF170, HSPA18, TFEB, SOX12, POLR3GL, MNAT1, TARS2, UBE2K, PLK1S1, DLST, MYH9, C2orf69, GLA, RAB8A, SFMBT2, NCAPH2, SVIP, TUBA4A, MSL3, WFS1, MRI1, ABHD2, CERK, KHK, LMF2, PTPN18, SLC1A4, CYTIP, ARSA, PRKD2, ARHGAP27, OBFC2A, LY96, SQRDL, AKNA, or GPSM3, or any combination thereof.

11. The method of claim 1 wherein the patient has not been treated with a proteasome inhibitor.

12. The method of claim 1 wherein the proteasome inhibitor comprises bortezamib, ixazanib, carfilzomib or oprozomib.

13. The method of claim 1 wherein the patient has relapsed or refractory myeloma that is not refractory to proteasome inhibitor treatment.

14. The method of claim 1 wherein the patient has not been treated for multiple myeloma.

15. The method of claim 1 wherein the combination of drugs includes a proteasome inhibitor.

16. The method of claim 1 wherein gene specific primers are employed to provide the profile.

17. The method of claim 1 wherein gene specific probes are employed to provide the profile.

18. A microarray having a plurality of probes or a composition having a plurality of primers for NEK3, DLST, PLK1S1, TARS2, C6orf48, HSPAIB, TFEB, RNF170, SOX12, ZNFX1-AS1, PAQR6, HIST1H2BD, UBE2K, LYSMD1, MNAT1, POLR3GL, SLCIA4, GLA, AKNA, ARHGAP27, LY96, MSL3, SQRDL, NCAPH2, MRI1, OBFC2A, RAB8A, ABHD2, LMF2, TUBA4A, C2orf69, PTPN8, PRKD2, KHK, CERK, GPSM3, WFS1, MYH9, CYTIP, SVIP, ARSA, SFMBT2, or any combination thereof.

19. The microarray or composition of claim 18 wherein the probes or primers include probes or primers for NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, POLR3GL, SLCIA4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, and SFMBT2.

20. The microarray or composition of claim 18 which does not include probes or primers specific for one, two, three, four, five, six, seven, eight, nine or ten genes selected from NEK3, DLST, PLK1S1, TARS2, C6orf48, HSPA1B, TFEB, RNF170, SOX12, ZNFX1-AS1, PAQR6, HIST1H2BD, UBE2K, LYSMD1, MNAT1, POLR3GL, SLC1A4, GLA, AKNA, ARHGAP27, LY96, MSL3, SQRDL, NCAPH2, MRI1, OBFC2A, RAB8A, ABHD2, LMF2, TUBA4A, C2orf69, PTPN18, PRKD2, KHK, CERK, GPSM3, WFS1, MYH9, CYTIP, SVIP, ARSA, or SFMBT2.
